(19) United States
(12) Patent Application Publication
(10) Pub. No.: US 2022/0323910 A1
(43) Pub. Date: Oct. 13, 2022

(54) SYSTEMS AND METHODS FOR TANGENTIAL FLOW DEPTH FILTRATION

(71) Applicant: Repligen Corporation, Waltham, MA (US)

(72) Inventors: Michael Bransby, Altadena, CA (US); Philip Yuen, Long Beach, CA (US); Rene Daniel Jean-Marie Gantier, Waltham, MA (US); David Bianchi, Waltham, MA (US); Jonathan Kuhlmann, Waltham, MA (US)

(73) Assignee: Repligen Corporation, Waltham, MA (US)

(21) Appl. No.: 17/719,850

(22) Filed: Apr. 13, 2022

Related U.S. Application Data
(60) Provisional application No. 63/174,418, filed on Apr. 13, 2021.

(51) Int. Cl. B01D 69/08; B01D 69/02; B01D 63/04

(52) U.S. Cl. CPC B01D 69/08; B01D 69/02; B01D 63/04; B01D 2325/023; B01D 2325/04; B01D 2313/243; B01D 2315/10

(57) ABSTRACT

The present disclosure relates to hollow fiber tangential flow filter units, including hollow fiber tangential flow depth filter units, for various applications, including bioprocessing and pharmaceutical applications, systems employing such filters, and methods of filtration using the same.

### CROSS REFERENCE TO RELATED APPLICATIONS

This application claims benefit of U.S. Provisional Application No. 63/174,418 filed on Apr. 13, 2021, the entire contents of which is incorporated by reference herein.

### FIELD OF THE DISCLOSURE

The present disclosure relates to systems and methods for filtration, particularly to tangential flow depth filtration (TFDF).

### BACKGROUND

Filtration is typically performed to separate, clarify, modify and/or concentrate a fluid solution, mixture or suspension. In the biotechnology and pharmaceutical industries, filtration is vital for the successful production, processing, and testing of new drugs, diagnostics and other biological products. For example, in the process of manufacturing recombinant proteins using animal or microbial cell culture, filtration is done for clarification, selective removal and concentration of certain constituents from the culture media or to modify the media prior to further processing. Filtration may also be used to enhance productivity by maintaining a culture in perfusion at high cell concentration.

Tangential flow filtration (also referred to as cross-flow filtration or TFF) systems are widely used in the separation of particulates suspended in a liquid phase, and have important bioprocessing applications. In contrast to dead-end filtration systems in which a single fluid feed is passed through a filter, tangential flow systems are characterized by fluid feeds that flow across a surface of the filter, resulting in the separation of the feed into two components: a permeate component which has passed through the filter and a retentate component which has not. Compared to dead-end systems, TFF systems are less prone to fouling. Fouling of TFF systems may be reduced further by alternating the direction of the fluid feed across the filtration element as is done in the XCell™ alternating tangential flow (ATF) technology commercialized by Repligen Corporation (Waltham, Mass.), by backwashing the permeate through the filter, and/or by periodic washing.

Modern TFF systems frequently utilize filters comprising one or more tubular filtration elements, such as hollow-fibers or tubular membranes. Where tubular filtration elements are used, they are typically packed together within a larger fluid vessel, and are placed in fluid communication with a feed at one end and at the other end with a vessel or fluid path for the retentate; the permeate flows through pores in the walls of the fibers into the spaces between the fibers and within the larger fluid vessel. Tubular filtration elements provide large and uniform surface areas relative to the feed volumes they can accommodate, and TFF systems utilizing these elements may be scaled easily from development to commercial scale. Despite their advantages, TFF systems filters may foul when filter flux limits are exceeded, and TFF systems have finite process capacities. Efforts to increase process capacities for TFF systems are complicated by the relationship between filter flux and fouling.

The Applicant, Repligen Corporation (Waltham, Mass.) has recently commercialized a new technology which overcomes some of the challenges affecting TFF systems. Tangential flow depth filtration (TFDFTM) utilizes thick-walled hollow-fiber filter elements. TFDF flowpaths are superficially similar to conventional TFF systems, but the filters themselves may differ in key ways, including fiber thickness and pore structure, which are described in greater detail herein.

The Applicant and others have demonstrated that TFDF supports viral production in continuous and batch cultures at very high-cell densities and permits rapid clarification and harvest at substantially higher fluxes than standard TFF filters. (See, e.g., Williams, et al. Cell & Gene Therapy Insights 2020; 6(3), 455-467; DOI: 0.18609/cgti.2020.053) These findings suggest the promise that TFDF holds for bioprocessing applications, but there is an ongoing need to adapt TFDF systems and methods to the diverse production, clarification and harvest needs of the industry.

### SUMMARY

In one aspect of the present disclosure, a hollow fiber tangential flow filter unit for bioprocessing applications comprises a housing having an interior, a fluid inlet, a retentate fluid outlet, a permeate fluid outlet, and at least one hollow fiber. The hollow fiber may comprise a porous wall that is formed from a plurality of extruded polymer filaments. The at least one hollow fiber may have an interior surface, an exterior surface, and a wall thickness. The wall thickness may range from 1 to 10 mm. The interior surface may form an interior lumen having a width ranging from 0.75 mm to 30.0 mm. The interior surface may extend though the at least one hollow fiber. The at least one hollow fiber may have a mean pore size ranging from 5-150 microns. The hollow fiber maybe positioned in the housing interior. The fluid inlet and the retentate fluid outlet may be in fluid communication with the interior lumen of the at least one hollow fiber. The permeate fluid outlet may be in fluid communication with the housing interior and the exterior surface of the porous wall.

In the above and other aspects, the extruded polymer filaments may comprise mono-component filaments. The at least one hollow fiber may comprise two or more layers of varying densities. The interior lumen may comprise a cross-section with a non-circular perimeter. The extruded polymer filaments may comprise bi-component filaments. The bi-component filaments may comprise a polyolefin and a polyester. The bi-component filaments may comprise polypropylene and polyethylene terephthalate. The extruded polymer filaments may be melt-blown filaments. A plurality of the extruded polymer filaments may be bonded to one another at spaced apart points of contact to define the porous wall. A plurality of the extruded polymer filaments may be thermally bonded to one another at spaced apart points of contact to define the porous wall. The hollow fiber may be formed by (a) assembling the extruded polymer filaments into a tubular shape and (b) heating the extruded polymer filaments such that the extruded polymer filaments become bonded to one another. The wall thickness may range from 2 to 3 mm, or from 2 to 8 mm. In some embodiments, the wall thickness may be about 5 mm. The at least one hollow fiber may have a mean pore size ranging from 20-100 microns. The at least one hollow fiber may have a mean pore size ranging from 15-50 microns. The at least one hollow fiber may have a mean pore size ranging from 20-40 microns. The interior lumen may have a width ranging from 1 to 2 mm, or from 2 to 10 mm, or from 5 to 20 mm. In some embodiments, the interior lumen may have a width of about 5 mm. The hollow fiber tangential flow filter unit may comprise a plurality of said hollow fibers. The hollow fiber tangential flow filter unit may comprise an inlet chamber positioned in an interior of the housing. The inlet chamber may be in fluid communication with the fluid inlet. The hollow fiber tangential flow filter unit may comprise an outlet chamber positioned in the interior of the housing. The outlet chamber may be in fluid communication with the retentate fluid outlet. The plurality of hollow fibers may extend between the inlet chamber and the outlet chamber. The inlet chamber and the outlet chamber may be in fluid communication with the interior lumen. The extruded polymer filaments may vary along a longitudinal length of at least one hollow fiber. The interior lumen may comprise one or more surface features. The interior lumen may comprise one or ridges, undulations, corners, or the like. The hollow fiber may comprise varying affinities along a longitudinal length thereof. A use of the hollow fiber tangential flow filter unit may include separating a fluid that comprises large size particles and small size particles into a permeate comprising the small size particles and a retentate comprising the large size particles. The fluid may further comprise intermediate-sized particles, which may be trapped in the wall of the at least one hollow fiber. The large particles may comprise eukaryotic and/or prokaryotic cells. The fluid may comprise a flocculant. The intermediate-sized particles may comprise cell debris. The small particles may comprise one or more of proteins, viruses, virus like particles (VLPs), exosomes, lipids, DNA, and cell metabolites.

A use of the hollow fiber tangential flow filter unit may comprise separating a fluid that comprises large size particles and intermediate size particles into a permeate comprising the intermediate size particles and a retentate comprising the large size particles. The fluid may further comprise small-sized particles, which may be trapped in the wall of the at least one hollow fiber. The large particles may comprise microcarriers or cell clumps. The intermediate particles may comprise cells. The intermediate particles may include but are not limited to mammalian cells. In some embodiments, the mammalian cells may comprise CHO cells or HEK293 cells. The fluid may comprise lysed cells, which may be lysed via chemical or mechanical means. The fluid may comprise small-sized particles that may be passed into the permeate. The small-size particles may comprise one or more of proteins, viruses, virus like particles (VLPs), exosomes, lipids, DNA, and cell metabolites.

A filtration method of using the hollow fiber tangential flow filter unit may comprise introducing a fluid that comprises large size particles and small size particles into the fluid inlet. The fluid may be separated into a permeate comprising the small particles. The permeate may exit the hollow fiber tangential flow filter unit through the permeate fluid outlet. A retentate may comprise the large particles. The retentate may exit the hollow fiber tangential flow filter unit through the retentate fluid outlet. The fluid further may comprise intermediate-sized particles. At least a portion of the intermediate-sized particles may be trapped in the wall of the hollow fiber tangential flow filter unit. The large particles may comprise microcarriers and/or cell clumps. The microcarriers may carry mammalian cells. The small particles may comprise one or more of proteins, viruses, virus like particles (VLPs), exosomes, lipids, DNA, and cell metabolites. The large particles and small particles may be of the same composition. The small particles may comprise colloidal flakes. The colloidal flakes may be separated out of the fluid based on addition of a flocculant to the fluid. The large and small particles may be selected from ceramic particles, metal particles, liposomal structures for drug delivery, biodegradable polymeric particles, and microcapsules. The large particles may comprise cells. The intermediate-sized particles may comprise cell debris. The small particles may comprise one or more of proteins, viruses, virus like particles (VLPs), exosomes, lipids, DNA, and cell metabolites. The large particles, the small particles and the intermediate-sized particles may be of the same composition. The large particles, the small particles and the intermediate-sized particles may be selected from ceramic particles, metal particles, liposomal structures for drug delivery, biodegradable polymeric particles, and microcapsules. The fluid may be introduced into the fluid inlet in a pulsed fashion.

A tangential flow filtering system may comprise a pump and the hollow fiber tangential flow filter unit.

A bioreactor system may comprise one or more of (a) a bioreactor vessel configured to contain bioreactor fluid, (b) a tangential flow filtering system comprising a pump and the hollow fiber tangential flow filter unit, and/or (c) a control system. The bioreactor vessel may have a bioreactor outlet. The bioreactor may have a bioreactor inlet. The bioreactor outlet may be in fluid communication with the fluid inlet. The bioreactor inlet may be in fluid communication with the retentate outlet.

The bioreactor fluid may comprise mammalian cells or bacterial cells. The control system may be configured to operate the pump such that a first flow of bioreactor fluid is pumped from the bioreactor outlet and into the fluid inlet, thereby separating the first flow of bioreactor fluid into a retentate flow and a permeate flow. The retentate flow may be re-circulated from the retentate outlet and into the bioreactor inlet. The permeate flow may be collected from the permeate fluid outlet. The system may be configured to pump the first flow of bioreactor fluid in a pulsed fashion.

In another aspect according to the present disclosure, a hollow fiber tangential flow filtering system may comprise a housing having an interior, a fluid inlet, a retentate fluid outlet, a permeate fluid outlet, and at least one hollow fiber. The at least one hollow fiber may comprise a porous wall that has an interior surface, an exterior surface, and a wall thickness. The wall thickness may range from 1 to 10 mm. The interior surface may form an interior lumen having a width ranging from 0.75 mm to 30.0 mm. The interior lumen may extend though the at least one hollow fiber. The at least one hollow fiber may have a mean pore size ranging from 5-150 microns. The at least one hollow fiber may be positioned in the housing interior. The fluid inlet and the retentate fluid outlet may be in fluid communication with the interior lumen of the at least one hollow fiber. The permeate fluid outlet may be in fluid communication with the housing interior and the exterior surface of the porous wall. The hollow fiber tangential flow filtering system may further comprise a pumping system configured to provide pulsed flow to the fluid inlet.

According to the above and other aspects of the disclosure, the pumping system may comprise a pulsatile pump. The pulsatile pump may be a peristaltic pump. The pumping system may comprise a pump and a flow controller that causes the pump to provide said pulsed flow. The flow controller may comprise an actuator configured to periodically restrict flow entering and/or exiting the pump. The flow controller may thereby provide pulsed flow to the fluid inlet. The flow controller may be positioned at the pump inlet. The actuator may be selected from an electrically controlled actuator, a pneumatically controlled actuator, or a hydraulically controlled actuator. The flow controller may comprise a servo valve or a solenoid valve. The flow controller may be configured to provide a pulsed flow having a flow rate that is pulsed at a rate ranging from 1 cycle per minute to 1000 cycles per minute. The pulsatile pump may comprise a pulse rate configurable in response to a change in viscosity in the fluid. The at least one hollow fiber may comprise a porous wall that is formed from a plurality of particles and/or filaments that are bonded together. The plurality of particles and/or filaments may be thermally bonded together.

A bioreactor system may comprise (a) a bioreactor vessel configured to contain bioreactor fluid, and (b) the hollow fiber tangential flow filtering system of at least the above aspect. The bioreactor vessel may have a bioreactor outlet and a bioreactor inlet. The bioreactor outlet may be in fluid communication with the fluid inlet and the bioreactor inlet may be in fluid communication with the retentate outlet. The pumping system may be configured to provide pulsed flow of bioreactor fluid into the fluid inlet. The pumping system may thereby separate the pulsed flow of bioreactor fluid into a retentate flow and a permeate flow. The retentate flow may be re-circulated from the retentate outlet and into the bioreactor inlet. The permeate flow may be collected from the permeate fluid outlet.

According to a further aspect of the present disclosure, a method of filtering a fluid comprising large size particles and small size particles may use a hollow fiber tangential flow filter unit including (a) a housing having an interior, (b) a fluid inlet, (c) a retentate fluid outlet, (d) a permeate fluid outlet, and (e) at least one hollow fiber. The at least one hollow fiber may comprise a porous wall that has an interior surface, an exterior surface, and a wall thickness. The thickness may range from 1 to 10 mm. The interior surface may form an interior lumen having a width ranging from 0.75 mm to 30.0 mm. The interior lumen may extend though the at least one hollow fiber. The at least one hollow fiber may be positioned in the housing interior. The fluid inlet and the retentate fluid outlet may be in fluid communication with the interior lumen of the at least one hollow fiber. The permeate fluid outlet may be in fluid communication with the housing interior and the exterior surface of the porous wall. The method may comprise pumping a pulsed flow of the fluid into the fluid inlet, thereby separating the fluid into a retentate flow comprising the large particles and a permeate flow comprising the small particles. The pulse flow may be set at a pulse rate based on the viscosity of the fluid.

In the above and other aspects of the present disclosure, the pulsed flow may have a flow rate that is pulsed at a rate above 1000 cycles per minute. The at least one hollow fiber may comprise a porous wall. The porous wall may be formed from a plurality of particles and/or filaments that are bonded together. The porous wall may be formed from a plurality of particles and/or filaments that are thermally bonded together. The fluid may comprise intermediate-sized particles, which may be trapped in the wall of the at least one hollow fiber. The large particles may comprise eukaryotic or prokaryotic cells. The intermediate-sized particles may comprise cell debris. The small particles may comprise one or more of proteins, viruses, virus like particles (VLPs), exosomes, lipids, DNA, and/or cell metabolites. The fluid may be fluid from a bioreactor. The retentate flow may be circulated back into the bioreactor. The pulse flow may be set at a pulse rate based on a measurement of a change in viscosity by a viscosity sensor (viscometer).

In another aspect, a hollow fiber tangential flow filtering system may comprise a housing having an interior, a fluid inlet, a retentate fluid outlet, a permeate fluid outlet, and at least one hollow fiber. The hollow fiber may comprise a porous wall that has an interior surface, an exterior surface, and a wall thickness ranging from 1 to 10 mm. The interior surface may form an interior lumen having a width ranging from 0.75 mm to 30.0 mm. The interior surface may extend though the at least one hollow fiber. The at least one hollow fiber may have a mean pore size ranging from 5-150 microns. The at least one hollow fiber may be positioned in the housing interior. The fluid inlet and the retentate fluid outlet may be in fluid communication with the interior lumen of the at least one hollow fiber. The permeate fluid outlet may be in fluid communication with the housing interior and the exterior surface of the porous wall. The hollow fiber tangential flow filtering system may include a pumping system that is configured to provide pulsed flow to the fluid inlet at a rate based on the viscosity of the fluid.

In at least the above aspect, the hollow fiber tangential flow filtering system may comprise a controller. The hollow fiber tangential flow filtering system may comprise a viscosity sensor communicatively coupled to the controller. The controller may be configured to adjust the rate of the pulsed flow based on a measurement of the viscosity sensor.

According to at least one additional aspect, a method of harvesting biological material from lysed cells may comprise measuring the viscosity of a fluid comprising the lysed cell and calculating a flow rate based on the measurement and a width of a hollow fiber of a tangential flow depth filter. The method may include passing the fluid alongside an outer surface of the hollow fiber at the flow rate. The method may include harvesting the biological material from a permeate collected from the fluid through the hollow fiber.

According to this and other aspects, the biological material may comprise viral vectors. The biological material may comprise plasmid DNA. The biological material may comprise at least one intracellular protein. The method may further comprise measuring a second viscosity of the fluid. The method may further comprise adjusting the flow rate based on the second viscosity.

In yet another aspect according to the disclosure, a potting gasket may comprise a molded silicone component defining at least one hole configured to couple with at least one corresponding hollow fiber and a peripheral flange configured to couple with a filter housing.

In the above and other aspects of the disclosure, the potting gasket may be configured to couple with the filter housing by forming an interference fit with a flange of the filter housing. The potting gasket may be configured to couple with the filter housing by forming an interference fit with a flange of a filter end cap. The potting gasket may be compressible between the flange of the filter housing and the flange of the filter end cap. The potting gasket may be compressible between the flange of the filter housing and the flange of the filter end cap based on a tightening of a clamp ring about an outer surface of the flange of the filter housing and the flange of the filter end cap.

A method of replacing the potting gasket may comprise removing a clamp ring from a filter end cap of a filter unit, thereby removing pressure from the gasket, removing the filter end cap, and removing the gasket, optionally together with one or more hollow fibers coupled thereto.

In the above and other aspects of the present disclosure, the culture medium may comprise a mammalian cell culture or a bacterial cell culture.

In an additional aspect, a method may include flaking out a plurality of suspended particles in a culture medium by adding a flocculant to the culture medium before or after lysis of one or more cells in the culture medium and adjusting a flux of the culture medium based on adding the flocculant.

In the above and other aspects of the present disclosure, the culture medium may comprise a mammalian cell culture or a bacterial cell culture.

In yet another aspect, a method of scaling a filter system may comprise replacing a first potting gasket with a second potting gasket, the second potting gasket configured to fit to a different number of hollow fibers than the first potting gasket.

In the above and other aspects, the potting gasket may comprise a molded silicone member. The potting gasket may comprise a peripheral flange configured to form an interference fit with a housing of the filter system, an end cap of the filter system, or both. The first potting gasket may be configured to be coupled with a first set of hollow fibers. The second potting gasket may be configured to be coupled with a second set of hollow fibers.

According to an at least one additional aspect, a system may comprise a filter housing, a filter end cap, and a silicone potting gasket disposed therebetween. The potting gasket may be configured to hold one or more hollow fibers defining permeate channels residing within a retentate chamber of the filter housing. The hollow fibers may open to a permeate chamber of the filter end cap. The silicone potting gasket may be configured to form an interference fit with the filter housing, the filter end cap, or both.

In at least this aspect, the system may comprise a clamp ring configured to fit to an outer surface of one or both of the filter housing and filter end cap. The clamp ring may be configured to affect a decrease in distance between the filter housing and the filter end cap, thereby compressing the silicone potting gasket. An outer surface of the filter end cap may comprise a curve or angle. The clamp ring may comprise a curved or angled surface configured to accommodate the curve or angle of the filter end cap.

In yet another aspect, a method of harvesting an intracellular product may comprise detecting a first viscosity measurement of a cell culture medium comprising at least one cell containing the intracellular product, lysing the at least one cell containing the intracellular product, detecting a second viscosity measurement of the cell culture medium, and adjusting a turbulence of a flow of the cell culture medium through a hollow fiber filter based on a difference between the first viscosity measurement and the second viscosity measurement.

According to the above and other aspects, the intracellular product may comprise AAV particles. The at least one cell may comprise a HEK293 cell. Adjusting the turbulence may comprise automatically effecting a flow rate of the cell culture medium via a controller communicably coupled to a pump.

### BRIEF DESCRIPTION THE DRAWINGS

The above and other aspects of the present disclosure will be more apparent from the following detailed description, presented in conjunction with the following drawings wherein:

FIG. 1A is a schematic cross-sectional view of a hollow fiber tangential flow depth filter according to the present disclosure;

FIG. 1B is a schematic partial cross-sectional view of three hollow fibers within a tangential flow filter like that shown in FIG. 1A.

FIG. 2 is a schematic cross-sectional view of a wall of a hollow fiber within a tangential flow depth filter like that shown in FIG. 1A.

FIG. 3 is a schematic illustration of a bioreactor system according to the present disclosure.

FIG. 4A is a schematic illustration of a disposable portion of a tangential flow filtering system according to the present disclosure.

FIG. 4B is a schematic illustration of a reusable control system according to the present disclosure.

FIG. 5 illustrates an example of an anisotropic filter according to one or more embodiments described herein.

FIGS. 6A-6F illustrate various cross-sections of hollow fibers according to various embodiments described herein.

FIG. 7 illustrates a bioreactor feed under a turbulent flow regime according to one or more examples as described herein.

FIG. 8 illustrates harvesting of cells from a bioreactor feed according to one or more embodiments described herein.

FIG. 9A illustrates a gasket according to one or more embodiments described herein.

FIG. 9B illustrates a gasket and filter housing according to one or more embodiments described herein.

FIG. 10A illustrates an alternative gasket according to one or more embodiments described herein.

FIG. 10B illustrates a cross-sectional view of the alternative gasket of

FIG. 10A with a filter housing according to one or more embodiments described herein.

FIG. 10C illustrates a gasket together with a filter housing, filter end cap, and clamp ring according to one or more embodiments described herein.

FIG. 11 is a bar graph illustrating the passage of CHO cells through TFDF filters of differing densities. For Trials 1-4, 5-7 and 8-10, the TFDF filter density was 35%, 25% and 53%, respectively.

### DETAILED DESCRIPTION

### Overview

The present disclosure describes new devices, systems, and methods for harvesting and/or perfusing biological material. Various embodiments of the present disclosure include tangential flow filters, and in particular hollow fiber (HF) tangential flow filters. Tangential flow filtration has traditionally been used in various applications of separation and purification of biomolecules in a range of fields such as immunology, protein chemistry, molecular biology, biochemistry, and microbiology.

However, presently disclosed embodiments may enable one or more benefits over traditional systems, for example, increasing an efficiency of harvesting and/or perfusion methods, enabling new methods of harvesting and/or separating biological material from cell culture, increasing the viable cell densities achievable in perfusion and batch cultures, and the like.

By way of example and without wishing to be bound by any particular theory, the capacity (e.g., as measured by maximum filter flux) and/or performance (e.g., as measured by the reduction of filter flux or increase of back-pressure in the feed channel over time) of thick-walled hollow fibers and TFDF units incorporating them may be proportional to the surface area of the hollow fiber that is available to contact a fluid of interest, and may be increased or decreased by varying the relative inner surface area of the thick-walled hollow fibers utilized in an “inside-out” TFDF system in which the fluid is fed into the lumen of the fiber(s) and permeate passes through the wall of the fiber(s) to an external space in which the fiber(s) are at least partially disposed.

Thus, certain embodiments of the of the present disclosure comprise thick-walled hollow fibers having inner and/or outer surfaces comprising features that increase or decrease the surface area available to interact with a fluid feed through the lumen of the fiber. Structural features that increase the available surface area can include, without limitation, one or more pleats, undulations, ridges, bumps, pits, and the like, which may extend along a partial or full longitudinal length of the hollow fiber surface. For example, hollow fibers may comprise internal and/or external surfaces which are polygonal, star-shaped, waved, or otherwise non-uniform along a perimeter thereof. Surface features of various embodiments of the present disclosure may be created via an extrusion process used to form the hollow fiber. For example, a hollow fiber with an internal star-shaped surface may be formed by an extrusion process in a single step. Alternatively, or additionally, surface features may be formed after an extrusion process. For example, a hollow fiber may be extruded with an excess diameter, which may then be cut away to form one or more surface features. Additive and/or subtractive processes may be used to generate one or more surface features according to the present disclosure. Embodiments are not limited in this context. Alternatively, or additionally, the area of the inner surface of a fiber may be reduced by eliminating surface heterogeneities such as those described above, or by coating at least a portion of the inner surface of a fiber with a material exhibiting a reduced porosity or reduced pore size relative to the unprocessed inner surface of the fiber.

Generally, thick-walled hollow-fibers described for use in tangential flow depth filtration (TFDF) are “isotropic” in the sense that their chemistry and pore structure (while heterogeneous), is substantially similar along their length and width. However, in some embodiments, anisotropy or heterogeneity in chemistry or physical characteristics may be desirable to improve performance or impart new characteristics. As one non-limiting example, a fiber of the present disclosure may comprise a surface (e.g., an inner wall surface, a pore surface) that is functionalized to permit non-covalent (e.g., ionic) bonding, or covalent cross-linking or conjugation to a functional moiety such as an ion-exchange or affinity ligand, or to a constituent of a fluid to be filtered. This may be achieved by any suitable method, including without limitation incorporation of various layers and/or anisotropic chemistries, which may customize and/or improve affinity and/or efficiencies of filtering systems over traditional systems.

For example, layers of silica and/or diatomaceous earth may retain negatively charged cell debris from a cell culture as the cell culture is passed through a hollow fiber. In another example, a membrane with a smaller pore diameter than the hollow fiber may be added to an external and/or internal wall of the hollow fiber. Accordingly, a turbidity of a culture medium passed through a wall of the hollow fiber may decrease in turbidity as it flows through the wall.

In some embodiments, a hollow fiber may be created with anisotropic properties along a longitudinal length thereof. For example, a first end may comprise a first porosity and a second end may comprise a second porosity, thereby enabling progressive passage of differently sized products along the length thereof In another example, a first end may comprise a first charged layer and a second end may comprise a second charged layer, thereby enabling progressive passage of differently charged products along the length thereof. Various membranes, layers, or the like may be distributed throughout a hollow fiber in distinct and/or in gradually intermediate sections.

Anisotropic hollow filter elements as disclosed herein may, in some embodiments, combine effects of various filtering elements into a single unit, reducing a footprint of TFF/ATF systems. In some embodiments, several filtering steps may be combined into a single step, for example, by combining various hollow fiber layers and/or filtering a fluid along a length of a hollow fiber varying in length. In various embodiments, at least one end of a hollow fiber may be closed, for example, as in a 3D depth filter.

In some embodiments according to the present disclosure, devices, systems, and/or methods for large pore tangential flow depth filtration (LPTFDF) may enable improved and/or new harvesting methods, such as of mammalian cells and/or improved cell sorting methods, such as of differentiating and/or otherwise growing cells.

Previously, methods of cell harvesting have been limited to small scales and batches (e.g., methods such as centrifugation, cell detachment, biopsies and other direct harvesting methods, or the like). Methods of harvesting using depth filtration have subject to fouling. As traditional TFF/ATF systems are configured to harvest permeates with contents smaller than typical sizes of mammalian cells (e.g., viruses, proteins, or the like, which may, for example, have diameters between 5 and 500 nanometers while mammalian cells may have diameters between 10 and 100 microns, although red blood cells may have diameters around 8 microns), TFF/ATF systems have not been traditionally useful for enabling scaling of entire harvesting methods. However, it is presently found that increasing a pore size of filters may enable TFF/ATF systems to be used to separate full mammalian cells from a cell culture media. For example, pores or presently disclosed filters may include pores with diameters in ranges of 5-120 microns, 10-100 microns, 10-50 microns, 20-40 microns, or the like.

Increasing a pore size of TFF/ATF systems may alternatively, or additionally, enable TFF/ATF systems to be used in methods of selectively harvesting cells of particular sizes and/or growth stages. Generally, methods of cell sorting such as fluorescent activated cell sorting (FACS) and magnetic activated cell sorting (MACS), buoyancy activated cell sorting (BACS), are expensive, while sorting with microfluidic devices, and/or single cell sorting are suitable only for processing of very small volumes of cell culture. Cell sorting has thus been difficult, if not impossible, to scale to industrial capacities. However, LPTFDF may allow a harvesting of individual cells into a permeate while cell clumps and/or microcarriers carrying growing cells are maintained in the retentate. It will be recognized that, furthermore, cells may peel off of cell clumps or microcarriers as they differentiate and grow, at which point they may be collected into a retentate via LPTFDF. Accordingly, cells may be grown and directly harvested from the same bioreactor without an intermediate need for detachment from microcarrier scaffolding, washing, or concentration of cells prior to a harvesting step. LPTFDF may thus present an economical and efficient process for selectively isolating individual cells, for example, according to a state of their differentiation. Devices and methods of the present disclosure may be easily scaled, for example, to process culture volumes of 10 L, 50 L, 100 L, 200 L, 500 L, 1000 L, 2000 L, 5000 L, or any volume in between.

Additionally, or alternatively, various examples of the present disclosure may present improvements of methods and devices for harvesting and/or perfusion of biological matter from cultures with high viscosity resulting from lysing of cells to release extracellular material. For example, cells within cultures may be lysed in order to release intracellular material for harvest, such as in AAV harvesting from HEK293 cells as is known in the art, for example, in vaccine production procedures. However, cell lysis may further release cell debris such as DNA, breached cell membranes, or the like, which may increase the viscosity of the culture and increase a fouling rate during filtering.

Various embodiments may enable harvesting of biological material from highly turbid cultures, for example, including lysed cells and the contents thereof, or culture medium containing flocculant and resultant colloidal flakes, by leveraging targeted levels of turbulent and/or non-laminar flow during filtering. In some examples, the viscosity of a culture may be measured, and, in response, a non-laminar flow rate of the culture may be adjusted so as to improve a filtering rate thereof. Embodiments are not limited in this context.

Some embodiments may employ improved devices, systems, and/or methods of potting hollow fibers as described herein. Traditional potting methods may permanently and/or irreversibly couple hollow fibers with respect to each other and/or a filter housing. Each filter housing unit, therefore, may traditionally be used for a single scale of process. However, according to the present disclosure, one or more hollow fibers may be encased or potted in a gasket, such as a molded silicone gasket. The gasket may be optionally removably coupled with a filter housing, a filter end cap, or both, for example, by an interference fit. In some embodiments, a clamp ring may secure the filter housing, end cap, and/or gasket with respect to each other, thereby improving an ability of the filter to withstand an internal pressure. The gasket may be replaceable, for example, with an alternative gasket configured to interface with a different number of hollow fibers. Accordingly, filtering systems may be easily scalable. In some embodiments, gaskets may double as sanitary gaskets, reducing an overall cost of filtering systems. Embodiments are not limited in this context.

Principles of the present disclosure may present novel improvements over traditional systems and/or methods whether taken alone or in any combination.

Accordingly, devices and systems of the present disclosure may be customized and/or optimized towards desired harvesting, perfusion, and/or sorting methods.

### Tangential Flow Filtering

Various traditional tangential flow filters (TFF) may be configured to harvest and/or purify biological products such as protein products, viruses, and other small-scale biological products. For example, filtration of biological fluids may be performed to separate blood into blood cells and plasma, among other implementations. For biomedical applications, it is important to be able to effectively carry out filtration so as to obtain precise amounts of desired material.

Alternating tangential flows of fluid may be used to perform filtering.

More particularly, by alternating a tangential flow of fluid through a filter element, continuous filtration may be achieved. An alternating tangential flow system is described, for example, in U.S. Pat. No. 6,544,424 to Shevitz, the entire contents of which are incorporated herein for the background, apparatuses and technical content therein. Generally, alternating tangential flow filtration (ATF) includes passing a fluid, such as a cell culture medium, across a membrane or other permeable structure in alternating directions. A portion of the volume of the fluid, comprising one or more components that are suspended or solved therewithin, passes through the membrane, creating a filtrate or permeate (which terms are used interchangeably here) in which one or more the suspended or solved components is present in a higher concentration than in the original fluid. The portion of the fluid volume that does not pass through the filter is referred to as the retentate, and the space comprising the retentate, and defined in part by the membrane, is referred to as the feed channel and/or the retentate chamber. The space on the opposite side of the membrane is referred to as the permeate or filtrate chamber.

Alternating flows across the membrane are generated by the alternating application of positive and negative pressure to a portion of the feed channel. Alternating pressure may be provided using any suitable means, including, without limitation, by a reversible pump such as a diaphragm pump, or a piston or plunger pump. Other pumps may include peristaltic or magnetic pumps that are reversible. Those of skill in the art will appreciate that suitable pump structures may be elements of other devices that are not dedicated to pumping or filtration. These other devices may include, without limitation, pipettors (also referred to as pipettes) and syringes, and may be actuated manually by a user, or automatically. These aspects of the disclosure are discussed in greater detail below.

Filtration may be performed for purification, selective removal of unwanted constituents, and/or for maintaining or perfusing cells in cell culture media at a high cell concentration. Various types of filters may be used, including hollow fiber filters containing a plurality of hollow fibers bundled together. Filtrating using the aforementioned alternating tangential flow may be carried out, for example, by larger-scale equipment such as pumps and centrifuges.

Various hollow fibers as described herein may be thick-walled hollow fibers. In various examples, a thick construction of the wall of a hollow fiber relative to other filters used in the art may allow for higher flow rates to be used. The thick construction may additionally, or alternatively, allow the hollow fiber to capture larger volumes of particulate before fouling. That is, the filter may have a higher “dirt loading capacity” relative to other filter elements used in the art. Dirt loading capacity may be defined as the quantity of particulate matter that can be trapped by a filter before a maximum allowable back pressure is reached.

### Description of Examples in Figures

Before turning to the figures, which illustrate the exemplary embodiments in detail, it should be understood that the present application is not limited to the details or methodology set forth in the description or illustrated in the figures. It should also be understood that the terminology is for the purpose of description only and should not be regarded as limiting. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, can be arranged, substituted, combined, and designed in a wide variety of different configurations, all of which are explicitly contemplated and made part of this disclosure.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e. g., “such as”) provided herein, is intended merely to better illuminate the embodiments and does not pose a limitation on the scope of the claims unless otherwise stated. No language in the specification should be construed as indicating any non-claimed element as essential.

Unless otherwise indicated, all numbers expressing quantities of properties, parameters, conditions, and so forth, used in the specification and claims are to be understood as being modified in all instances by the term “about.” Accordingly, unless indicated to the contrary, the numerical parameters set forth in the following specification and attached claims are approximations. Any numerical parameter should at least be construed in light of the number reported significant digits and by applying ordinary rounding techniques. The term “about” when used before a numerical designation, e.g., temperature, time, amount, and concentration including range, indicates approximations which may vary by (+) or (−) 10%, 5% or 1%, or within a range of 1.

As will be understood by one of skill in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member.

Furthermore, while description herein generally refers to use of hollow fiber filters, it will be understood that various embodiments, methods, or the like described herein may alternatively, or additionally, be applicable with respect to flat filter membranes. For example, multi-layered filter membranes as described with respect to FIG. 5 may be flat membranes. Embodiments are not limited in this context.

Description herein may refer to various types of filtration and/or filters, for example, ATF, TFF, LPTFDF, or the like. For the sake of brevity and without intent to limit, one will understand that methods and/or components as described herein with respect to one such filter type may alternatively or additionally be respectively applied to others.

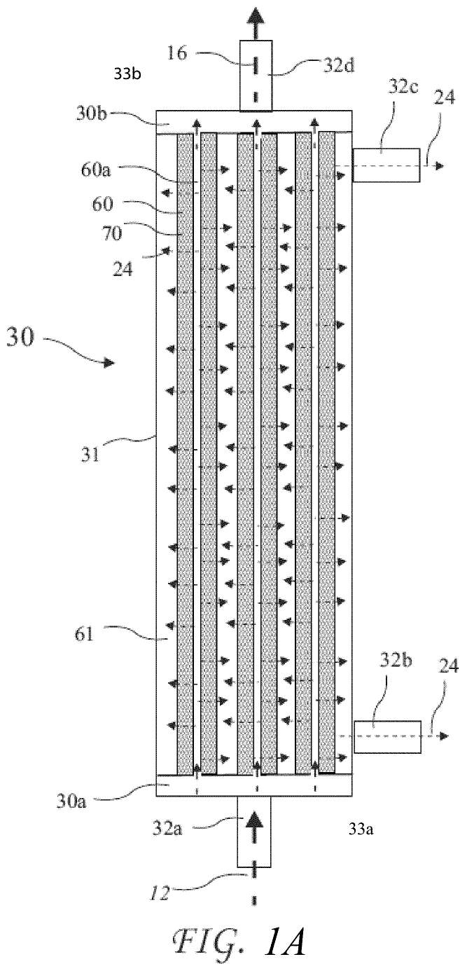

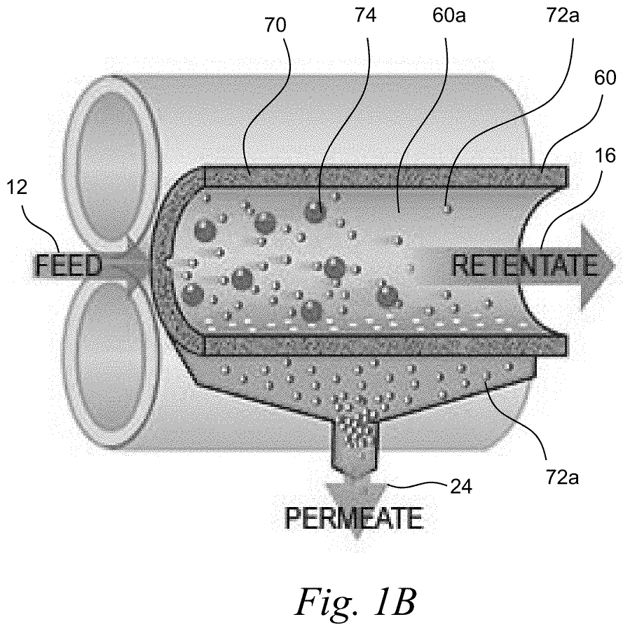

A schematic cross-sectional view of a hollow fiber tangential flow filter unit 30 in accordance with present disclosure is shown in FIG. 1A. The hollow fiber tangential flow filter unit 30 includes parallel hollow fibers 60 extending between an inlet chamber 30a and an outlet chamber 30b defined at ends of filter housing 31 by respective end caps 33a and 33b. A fluid inlet port 32a provides a flow 12 to the inlet chamber 30a and a retentate fluid outlet port 32d receives a retentate flow 16 from the outlet chamber 30b. The hollow fibers 60 receive the flow 12 through the inlet chamber 30a. The flow 12 is introduced into a hollow fiber interior 60a of each of the hollow fibers 60, and a permeate flow 24 passes through walls 70 of the hollow fibers 60 into a permeate chamber 61 within a filter housing 31. The permeate flow 24 travels to permeate fluid outlet ports 32b and 32c. Although two permeate fluid outlet ports 32b and 32c are employed to remove permeate flow 24 in FIG. 1A, in other embodiments, only a single permeate fluid outlet port may be employed. Filtered retentate flow 16 moves from the hollow fibers 60 into the outlet chamber 30b and is released from the hollow fiber tangential flow filter unit 30 through retentate fluid outlet port 32d.

FIG. 1B is a schematic partial cross-sectional view of three hollow fibers 60 within a hollow fiber tangential flow filter unit analogous to that shown in FIG. 1A, and shows the separation of an inlet flow 12 (also referred to as a feed) which contains large particles 74 and target particles 72a into a permeate flow 24 containing a portion of the small particles and a retentate flow 16 containing the large particles 74 and a portion of the target particles 72a that does not pass through the walls 70 of the follow fibers 60.

Tangential flow filters in accordance with the present disclosure include tangential flow filters having depths and mean pore sizes (for example, as defined by bubble point testing) that are suitable for applications discussed herein, for example, excluding large particles (e.g., cell clumps, microcarriers, or other large particles), trapping intermediate-sized particles (e.g., cell debris, or other intermediate-sized particles), and allowing passage therethrough of target particles (e.g., individual cells, cells with soluble and insoluble cell metabolites and other products produced by cells including expressed proteins, viruses, virus like particles (VLPs), exosomes, lipids, DNA, or other small particles). For example, a mean pore size of filters as described herein may range from 5 microns to 20 microns, or from 15 microns to 150 microns, or from 20 microns to 50 microns, or from 30 microns to 40 microns, or other inclusive range. Similarly, the TDF 100 may have any suitable wall thickness, e.g. between 0.1 mm and 10 mm, any suitable inner diameter, e.g., between 0.75 mm to 35.0 mm, such as 25.4 mm, and any suitable length, e.g., between 20-200 cm. As used herein a “microcarrier” is a particulate support allowing for the growth of adherent cells in bioreactors.

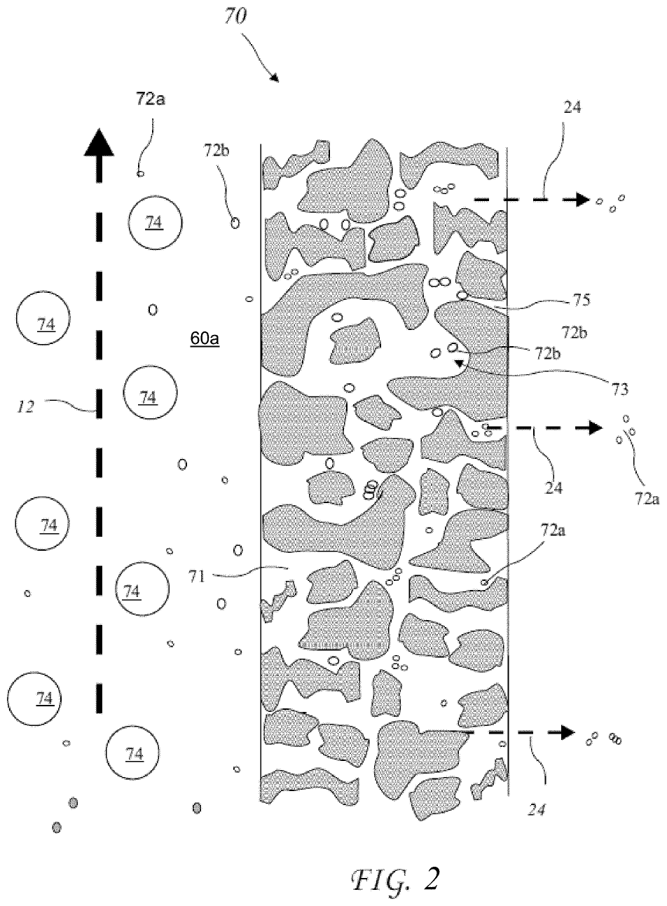

In this regard, FIG. 2 is a schematic cross-sectional illustration of a wall 70 of a hollow fiber 60 used in conjunction with a hollow fiber tangential flow filter unit 30 like that of FIG. 1A. In FIG. 2, a flow 12 comprising large particles 74, target particles 72a, and intermediate-sized particles 72b is introduced into the fluid inlet port 32a of the hollow fiber tangential flow filter unit 30. The large particles 74 pass along the inner surface of the wall 70 that forms the hollow fiber interior 60a (also referred to herein as the fiber lumen) of the hollow fibers and are ultimately released in the retentate flow. The wall 70 includes tortuous paths 71 that capture certain elements (i.e., intermediate-sized particles 72b) of the flow 12 as a portion of the flow 12 passes through the wall 70 of hollow fiber tangential flow filter unit 30 while allowing other particles (i.e., target particles 72a) to pass through the wall 70 as part of the permeate flow 24. In the schematic cross-sectional illustration of FIG. 2, settling zones 73 and narrowing channels 75 are illustrated as capturing intermediate-size particles 72b which enter the tortuous paths 71, while allowing smaller particles 72a to pass through the wall 70, thus trapping intermediate-size particles 72b and causing a separation of the intermediate-size particles 72b from smaller particles 72a in the permeate flow 24. This method is thus different from filtering obtained by the surface of standard thin wall hollow fiber tangential flow filter unit membranes, wherein intermediate-size particles 72b can build up at the inner surface of the wall 70, clogging entrances to the tortuous paths 71.

In this regard, one of the most problematic areas for various filtration processes, including filtration of cell culture fluids such as those filtered in perfusion and harvest of cell culture fluids, is decreased mass transfer of target molecule or particle due to filter fouling. The present disclosure overcomes many of these hurdles by combining the advantages of tangential flow filtration with the advantages of depth filtration. As in standard thin wall hollow fiber filters using tangential flow filtration, cells are pumped through the lumens of the hollow fibers, sweeping them along the surface of the inner surface of the hollow fibers, allowing them to be recycled for further production. However, instead of the protein and cell debris forming a fouling gel layer at the inner surface of the hollow fibers, the wall may add what is referred to herein as a “depth filtration” feature that traps the cell debris inside the wall structure, enabling increased volumetric throughput while maintaining close to 100% passage of typical target proteins in various embodiments of the disclosure. Such filters may be referred to herein as tangential flow depth filters (TFDF).

TFDF systems may be characterized by several filter parameters and operating variables. Filter parameters include TDF inner diameter (d), TDF length (1), TDF cross-sectional area (A) and number of TDF units in the filter (N). Operating variables include feed flow rate (Q_{F}), kinematic viscosity (μ), feed velocity per TDF (V_{F}), shear rate (γ) and Reynolds number (Re). Relations between filter parameters and operating variables are set forth in Table 1:

TABLE 1
Filter parameters and operating variable relations

| | |
|---|---|
| TDF cross-sectional area (A) | $\frac{π \cdot {d}^{2}}{4} \qquad [ 1 ]$ |
| Feed velocity per TDF (V_{F}) | ${V}_{F} = \frac{{Q}_{F}}{A \cdot N} \qquad [ 2 ]$ |
| Shear rate (γ) | $γ = \frac{8 \cdot {V}_{F}}{d} \qquad [ 3 ]$ |
| Reynolds number (Re) | $Re = \frac{{V}_{F} \cdot d}{μ} = \frac{γ \cdot {d}^{2}}{8 μ} \qquad [ 4 ]$ |

The Reynolds number is predictive of fluid flow behavior. When applied to fluid flows in tubular systems, laminar flows are expected where Reynolds numbers are below approximately 2300, turbulent are expected at Reynolds numbers above 4000, and a laminar-to-turbulent transition occurs between these values. While noting that laminar and turbulent flow behavior in TDFs may differ somewhat from the modeled behavior of non-permeable tubular systems, the inventors have found that TFDF systems according to this disclosure tolerate very high fluxes without fouling when the Reynolds number for the inlet flow 12 is above the laminar flow range, e.g., above approximately 2300, 2500, 3000, 3500, 4000, etc. While not wishing to be bound by any theory, it is believed that turbulent feed flows may generate enhanced particle transport from the wall 70 to the bulk flow through the hollow fiber 60, which may reduce fouling compared to laminar flows. Accordingly, various embodiments of this disclosure may be directed to methods of operating TFDF systems which utilize feed flows that are turbulent or within the laminar-to-turbulent transition region, e.g., characterized by Re values above 2000, 2300, 2500, 3000, 3500, 4000, etc. Because Re increases with increases in feed velocity, shear rate and/or TDF inner diameter, certain methods of this disclosure involve operating a TFDF system with feed velocities or shear rates selected to yield Re values above 2000, 2300, 2500, 3000, 3500, 4000, etc. Because dilute aqueous solutions have a kinematic viscosity of approximately 1 centistoke (cSt), certain embodiments of the method involve operating a TFDF system under conditions in which a product of the feed velocity and the TDF diameter is greater than 2000, 2300, 2500, 3000, 3500, or 4000 mm²s⁻¹. In other embodiments, the method involves operating a TFDF system under conditions in which the feed velocity is 2000, 2300, 2500, 3000, 3500 or 4000 times greater than the quotient of the kinematic viscosity over the TDF inner diameter

$$( \frac{μ}{d} ) ,$$

which is approximately equal to

$$\frac{1}{d}$$

for dilute aqueous solutions.

Additional embodiments of the disclosure are directed to TFDF systems configured for operation under conditions in which Re values exceed 2000, 2300, 2500, 3000, 3500, 4000, etc. In certain embodiments, a product of the feed velocity and the TDF diameter is greater than 2000, 2300, 2500, 3000, 3500, or 4000 mm²s⁻¹. Other embodiments of the disclosure relate to systems configured to operate under conditions in which the feed velocity is 2000, 2300, 2500, 3000, 3500 or 4000 times greater than the quotient of the kinematic viscosity over the TDF inner diameter.

Certain embodiments of this disclosure utilize TDF geometries that are selected to promote non-laminar flow. Increases in internal diameter, for example, will tend to promote more turbulent flows at the given shear rate. The TDFs used in the embodiments of the disclosure may have inner diameters greater than 1 mm and/or walls with a thickness greater than 0.1 mm to withstand operation under high-flux conditions. Systems and methods of this disclosure may be employed in alternating tangential flow (ATF) setups, or under a constant tangential flow, and any suitable pump technology may be employed to drive feed flows. The TDF walls may have a constant or variable density and, consequently, a constant or variable average and maximum pore diameter across their length and/or circumference. The porosity of the TDFs may be further controlled by applying a coating or coatings to TDF wall surfaces. Skilled artisans will appreciate that additional modifications of TDF surfaces may be possible, including without limitation the use of affinity reagents to selectively purify specific molecular species (e.g., protein A coatings may be used to pull down human IgG).

Those of skill in the art will appreciate that, for feed flows characterized by Reynolds number at or just above the transition value of about 2300, decreases in velocity over the length of the TDFs may result in flows below the 2300 Re transition value within the TDFs. In some examples, improvements in filter capacity and fouling behavior are observed at Re values at the feed as low as 2300, indicating that a turbulent flow does not necessarily need to be maintained throughout the length of the TDF, and that a turbulent flow across a portion of the length of the TDF may be sufficient to improve filter capacity and fouling behavior to some degree. Thus, in certain embodiments of the disclosure, a TFDF system is operated under conditions in which VF at the feed is between 2300 and 2500, or between 2300 and 3000. In some embodiments, a TFDF system is operated such that a turbulent flow is produced across a portion of the length of the TDF units in the filter.

In some embodiments, a flocculant may be added to a culture medium comprising eukaryotic and/or prokaryotic cells. It will be recognized that floc, or colloidal particles which have “flaked” from a culture medium based on an addition of flocculant, may foul traditional filtering systems. However, in some examples according to the present disclosure, a flux of a culture medium may be adjusted in coordination with an addition of flocculant to the culture medium. For example, an amount and/or type of flocculant may be considered in an algorithmic estimation of expected floc for a culture, which may be used to determine a change in flux which may be necessary to retain a fouling rate of a filter or maintain the fouling rate within a predetermined range. The turbulence and/or flow rate of the culture medium may accordingly be adjusted based on the determined change in flux. Accordingly, embodiments of the present disclosure may improve applications including addition of flocculant to culture medium by mitigating associated changes in risk of filter fouling.

In some examples, culture medium may be added to a bioreactor to replace a permeate volume based on a rate of permeate collection, for example, in order to maintain an overall volume of a cell culture. In various examples, surfactant may be added to the bioreactor with the replacement culture medium. The surfactant may further reduce a fouling risk for a TDF system while harvesting from lysed cells.

The TDF system may include a viscosity sensor and/or controller such that a desired turbulence calculation may be automated. In various embodiments, the TFF/ATF system may include a mixer and/or other means for effecting a turbulence of the culture medium. The mixer and/or other means for effecting the turbulence of the culture medium may be communicatively coupled to a controller used to calculate a desired turbulence and configured to adjust a turbulence of the culture medium based on the desired turbulence calculation. One of skill in the art will recognize that dynamic flow adjustment methods as described herein may enable TDF systems to be used to filter otherwise unacceptably viscous culture mediums, for example, with hollow fibers having pore densities such as 50%, 53%, 60%, or the like, although hollow fibers may alternatively, or additionally, have pore densities, such as discussed herein with respect to hollow fibers, such as those of large pore depth filtration tangential filters.

TFDF Systems according to the present disclosure may be used to filter a variety of fluids and separate a variety of soluble or particulate species. These include, without limitation, mammalian cells or other eukaryotic cells, microbial cells, including bacterial cells, such as E. coli, and/or synthetic nanoparticles, such as particles for drug delivery, as well as biomolecules such as polypeptides, polynucleotides, polysaccharides, and complexes of one or more of these. Without limiting the foregoing, the systems and methods of this disclosure can be used in the production and purification of recombinant proteins such as immunoglobins or functional fragments thereof. It will be recognized that various embodiments of the present disclosure may enable perfusion of, growth of, and harvesting from a cell culture from a same bioreactor. For example, HEK293 cells may be perfused, grown to a desired population, for example, 40 million, lysed, and their expressed protein harvested using a turbulent flow regime based on a dynamic viscosity of the cell medium. Those of skill in the art will appreciate that the systems and methods of this disclosure may be applied in any setting in which hollow-fiber TFF systems are currently used, such as clarifying animal or microbial cultures, concentration and fractionation of species such as those described above.

As illustrated schematically in FIG. 2, tangential flow depth filters in accordance with various embodiments of the present disclosure do not have a precisely defined pore structure. Particles that are larger than the “pore size” of the filter will be stopped at the surface of the filter. A significant quantity of intermediate-sized particles, on the other hand, enter the wall for the filter, and are entrapped within the wall before emerging from the opposing surface of the wall. Smaller particles and soluble materials can pass though the filter material in the permeate flow. Being of thicker construction and higher porosity that many other filters in the art, the filters can exhibit enhanced flow rates and dirt loading capacity.

Despite a lack of a precisely defined pore structure, the pore size of a given filter can be objectively determined via a widely used method of pore size detection known as the “bubble point test.” The bubble point test is based on the fact that, for a given fluid and pore size, with constant wetting, the pressure required to force an air bubble through a pore is inversely proportional to the pore diameter. In practice, this means that the largest pore size of a filter can be established by wetting the filter material with a fluid and measuring the pressure at which a continuous stream of bubbles is first seen downstream of the wetted filter under gas pressure. The point at which a first stream of bubbles emerges from the filter material is a reflection of the largest pore(s) in the filter material, with the relationship between pressure and pore size being based on Poiseuille's law which can be simplified to P=K/d, where P is the gas pressure at the time of emergence of the stream of bubbles, K is an empirical constant dependent on the filter material, and d is pore diameter. In this regard, pore sizes determined experimentally herein are measured using a POROLUX™ 1000 Porometer (Porometer NV, Belgium), based on a pressure scan method (where increasing pressure and the resulting gas flow are measured continuously during a test), which provides data that can be used to obtain information on the first bubble point size (FBP), mean flow pore size (MFP) (also referred to herein as “mean pore size”), and smallest pore size (SP). These parameters are well known in the capillary flow porometry art.

In various embodiments, hollow fibers for use in the present disclosure may have, for example, a mean pore size ranging from 5 microns (μm) or less to 50 microns or more, for example, 7 to 10 microns, 10 to 20 microns, or 20 to 30 microns, or 20 to 40 microns, or 30 to 50 microns, or 30 to 60 microns, or 40 to 60 microns, among other possible values, for example, up to 150 microns.

In various embodiments, the hollow fibers for use in the present disclosure may have, for example, a wall thickness ranging from 1 mm to 10 mm, for example, ranging from about 2 mm to about 3 mm, about 2 mm to about 8 mm, or about 5 mm, among other values.

In various embodiments, hollow fibers for use in the present disclosure may have, for example, an inside diameter (i.e., a lumen diameter) ranging from 0.75 mm to 30.0 mm, for example, ranging from 1 mm to 3.5 mm, from 2 mm to 10 mm, from 5 mm to 20 mm, among other values, such as about 4.6 mm, about 5 mm or about 25 mm. In general, a decrease in inside diameter will result in an increase in shear rate. Without wishing to be bound by theory, it is believed that an increase in shear rate may enhance flushing of cells and cell debris from the walls of the hollow fibers.

The hollow fibers for use in the present disclosure may be formed from a variety of materials using a variety of processes.

For example, hollow fibers may be formed by assembling numerous particles, filaments, or a combination of particles and filaments into a tubular shape. The pore size and distribution of hollow fibers formed from particles and/or filaments will depend on the size and distribution of the particles and/or filaments that are assembled to form the hollow fibers. The pore size and distribution of hollow fibers formed from filaments will also depend on the density of the filaments that are assembled to form the hollow fibers. For example, mean pore sizes ranging from 0.5 microns to 150 microns may be created by varying filament density, such as within inclusive ranges of 15 to 80 microns, 5 to 150 microns, 15 to 150 microns, 30 to 40 microns, 30 to 50 microns, 20 to 60 microns, 20 to 50 microns, 20 to 40 microns, or the like.

Suitable particles and/or filaments for use in the present disclosure include both inorganic and organic particles and/or filaments. In some embodiments, the particles and/or filaments may be mono-component particles and/or mono-component filaments. In some embodiments, the particles and/or filaments may be multi-component (e.g., bi-component, tri-component, etc.) particles and/or filaments. For example, bi-component particles and/or filaments having a core formed of a first component and a coating or sheath formed of a second component, may be employed, among many other possibilities.

In various embodiments, the particles and/or filaments may be made from polymers. For example, the particles and/or filaments may be polymeric mono-component the particles and/or filaments formed from a single polymer, or they may be polymeric multi-component (i.e., bi-component, tri-component, etc.) particles and/or filaments formed from two, three, or more polymers. A variety of polymers may be used to form mono-component and multi-component particles and/or filaments including polyolefins such as polyethylene and polypropylene, polyesters such as polyethylene terephthalate and polybutylene terephthalate, polyamides such as nylon 6 or nylon 66, fluoropolymers such as polyvinylidene fluoride (PVDF) and polytetrafluoroethylene (PTFE), among others.

Particles may be formed into tubular shapes by using, for example, tubular molds. Once formed in a tubular shape, particles may be bonded together using any suitable process. For instance, particles may be bonded together by heating the particles to a point where the particles partially melt and become bonded together at various contact points (a process known as sintering), optionally, while also compressing the particles. As another example, the particles may be bonded together by using a suitable adhesive to bond the particles to one another at various contact points, optionally, while also compressing the particles. For example, a hollow fiber having a wall analogous to the wall 70 that is shown schematically in FIG. 2 may be formed by assembling numerous irregular particles into a tubular shape and bonding the particles together by heating the particles while compressing the particles.

Filament-based fabrication techniques that can be used to form tubular shapes include, for example, simultaneous extrusion (e.g., melt-extrusion, solvent-based extrusion, etc.) from multiple extrusion dies, or electrospinning or electrospraying onto a rod-shaped substrate such as a mandrel (which is subsequently removed), among others.

Filaments may be bonded together using any suitable process. For instance, filaments may be bonded together by heating the filaments to a point where the filaments partially melt and become bonded together at various contact points, optionally, while also compressing the filaments. As another example, filaments may be bonded together by using a suitable adhesive to bond the filaments to one another at various contact points, optionally while also compressing the filaments.

In particular embodiments, numerous fine extruded filaments may be bonded together to at various points to form a hollow fiber, for example, by forming a tubular shape from the extruded filaments and heating the filaments to bond the filaments together, among other possibilities.

In some instances, the extruded filaments may be melt-blown filaments. As used herein, the term “melt blown” refers to the use of a gas stream at an exit of a filament extrusion die to attenuate or thin out the filaments while they are in their molten state. Melt-blown filaments are described, for example, in U.S. Pat. No. 5,607,766 to Berger. In various embodiments, mono- or bi-component filaments are attenuated as they exit an extrusion die using known melt blowing techniques to produce a collection of filaments. The collection of filaments may then be bonded together in the form of a hollow fiber.

In certain beneficial embodiments, hollow fibers may be formed by combining bicomponent filaments having a sheath of first material which is bondable at a lower temperature than the melting point of the core material. For example, hollow fibers may be formed by combining bicomponent extrusion technology with melt blown attenuation to produce a web of entangled biocomponent filaments, and then shaping and heating the web (e.g., in an oven or using a heated fluid such as steam or heated air) to bond the filaments at their points of contact. An example of a sheath-core melt blown die is schematically illustrated in U.S. Pat. No. 5,607,766 in which a molten sheath-forming polymer and a molten core-forming polymer are fed into the die and extruded from the same. The molten bicomponent sheath-core filaments are extruded into a high velocity air stream, which attenuates the filaments, enabling the production of fine bicomponent filaments. U.S. Pat. No. 3,095,343 to Berger shows an apparatus for gathering and heat-treating a multi-filament web to form a continuous tubular body (e.g., a hollow fiber) of filaments randomly oriented primarily in a longitudinal direction, in which the body of filaments are, as a whole, longitudinally aligned and are, in the aggregate, in a parallel orientation, but which have short portions running more or less at random in non-parallel diverging and converging directions. In this way, a web of sheath-core bicomponent filaments may be pulled into a confined area (e.g., using a tapered nozzle having a central passageway forming member) where it is gathered into tubular rod shape and heated (or otherwise cured) to bond the filaments.

In various embodiments, a density of the contact points throughout the hollow fiber may be customized in order to yield a particular pore density, mean pore size, or the like. For example, various embodiments according to these aspects of the disclosure may utilize hollow filters comprising sintered or melt-blown polymers, which hollow fibers optionally have densities that are 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59% or 60% of the density of an equivalent solid volume of the polymer. In some embodiments, the hollow fiber has a pore size or first bubble point size between 1 and 150 microns, and/or an inner diameter between 0.75 and 35.0 mm, such as 25.4 mm, and/or a length between 200 and 2000 mm.

Various materials and/or distributions of material(s) may be anisotropically or isotropically combined so as to customize one or more areas of a hollow fiber for specified filtering affinities. For example, one or more filaments may be applied in varying densities across a surface of a hollow fiber, or different types of filaments may be applied in concentric layers, or both. Such an example is illustrated in FIG. 5, in which wall 70 comprises three layers 82, 84, and 86. Each of layers 82, 84, and 86 may comprise the same or different materials than each other. In the non-limiting example of FIG. 5, each of layers 82, 84, and 86 may be seen to have varying mean pore sizes. For example, layer 84 may be seen to have a varying density along a longitudinal axis thereof (axis not shown for the sake of simplicity in the drawings), and in particular, a higher density at a top portion than at a bottom portion. Layer 86 may have a higher density than layer 82. In some examples, one or each of layers 82, 84, or 86 may comprise a different charge and/or chemical affinity to binding to one or more particles of bioreactor feed 12 (for example, one or more layers may comprise at least one affinity ligand, which may or may not be consistent across layers). For example, layer 84 may comprise a charge which provides it with a higher binding affinity to intermediate-sized particles 72b than layer 82, such as if layer 84 comprises diatomaceous earth or other positively charged component configured to bind negatively charged cell debris but allow other product (e.g., plasmid DNA, viral vectors, or intracellular proteins) to pass through.

It is presently contemplated that retentate-side walls of hollow fibers comprising lower densities may enable higher flux of fluid therethrough, which may in some examples lower a risk of filter fouling. Accordingly, an inner layer of a hollow fiber (e.g., layer 82) may be formed to be less dense than an outer layer, or an outer layer with a lower mean pore size than an inner layer may surround the inner layer. For example, an inner layer of a filter may comprise a mean pore size in the range of 1 to 150 microns, whereas an outer layer (e.g., layer 86) may comprise a mean pore size of 0.2 microns, 0.8 microns, or the like.

Anisotropic hollow fibers as described herein may be configured as depth filters, for example, dead end depth filters, enabling various layers of depth filter elements to interact differently with bioreactor fluid. It is presently contemplated that various embodiments of anisotropic hollow fibers may thus enable multiple filter steps to be effectively performed simultaneously. Embodiments are not limited in this context.

In certain examples, as-formed hollow fiber may be further coated with a suitable coating material (e.g., PVDF) either on the inside or outside of the fiber, which coating process may also act to reduce the pore size of the hollow fiber, if desired.

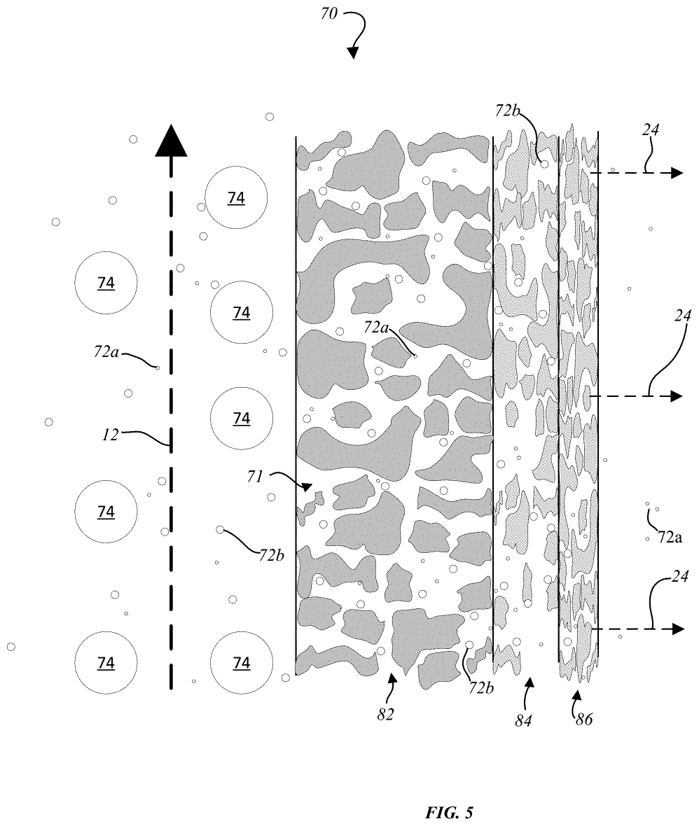

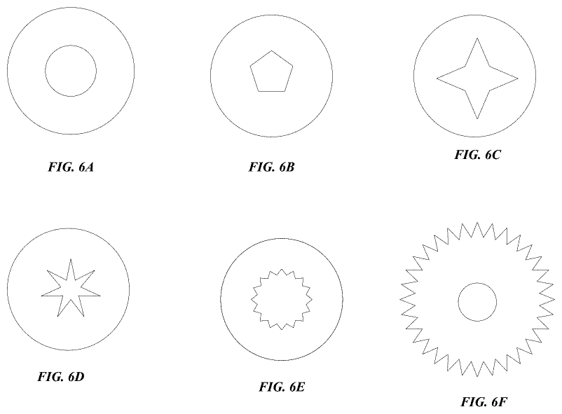

In some embodiments, hollow fibers may be formed with a variety of cross-sections, as illustrated in FIGS. 6A-6F. For example, hollow fibers may comprise cross-sections with circular perimeters of inner walls (as in FIG. 6A), polygonal inner walls with any number or configuration of sides (such as the polygonal inner wall of FIG. 6B), star-shaped walls with any number of ridges or edges (such as the 4-point star inner wall of FIG. 6C, the 7-point star inner wall of FIG. 6D, or the 16-point star inner wall of FIG. 6E), or the like. Alternatively, or additionally, outer walls of fibers may comprise non-circular cross-sectional shapes of any orientation as discussed with respect to inner walls (such as the multi-edged hollow fiber cross-section of FIG. 6F). Accordingly, surface area of hollow fibers may be adjusted to improve filtering efficiency therethrough. It will be further understood that cross-sectional shapes such as illustrated in FIGS. 6A-6F may extend fully or partially along a longitudinal length of a hollow fiber.

Hollow fibers such as those described above may be used to construct tangential flow filters for bioprocessing and pharmaceutical applications. Examples of bioprocessing applications in which such tangential flow filters may be employed include those where cell culture fluid is processed to separate cells from smaller particles such as proteins, viruses, virus like particles (VLPs), exosomes, lipids, DNA, other metabolites, or other cells.

Such applications include perfusion applications in which smaller particles are continuously removed from cell culture medium as a permeate fluid while cells are retained in a retentate fluid returned to a bioreactor (and in which equivalent volumes of media are typically simultaneously added to the bioreactor to maintain overall reactor volume). Such applications further include clarification or harvest applications in which smaller particles (typically biological products) are more rapidly removed from cell culture medium as a permeate fluid.

Hollow fibers such as those described above may be used to construct tangential flow depth filters for particle fractionation, concentration and washing. Examples of applications in which such tangential flow filters may be employed include the removal of small particles from larger particles using such tangential flow depth filters, the concentration of microparticles using such tangential flow depth filters, and washing microparticles using such tangential flow filters.

In some embodiments according to principles of the present disclosure, tangential flow filters may be formed including a plurality of hollow fibers, as discussed above, arranged alongside one another. In various embodiments, a plurality of hollow fibers may be arranged to be parallel or approximately parallel with each other. The plurality of hollow fibers may be permanently or removably secured in a position with respect to each other, a filter housing, or both at a top, bottom, or anywhere else along a longitudinal length of the hollow fibers, for example, by a potting, sealant, frame, gasket, or the like.

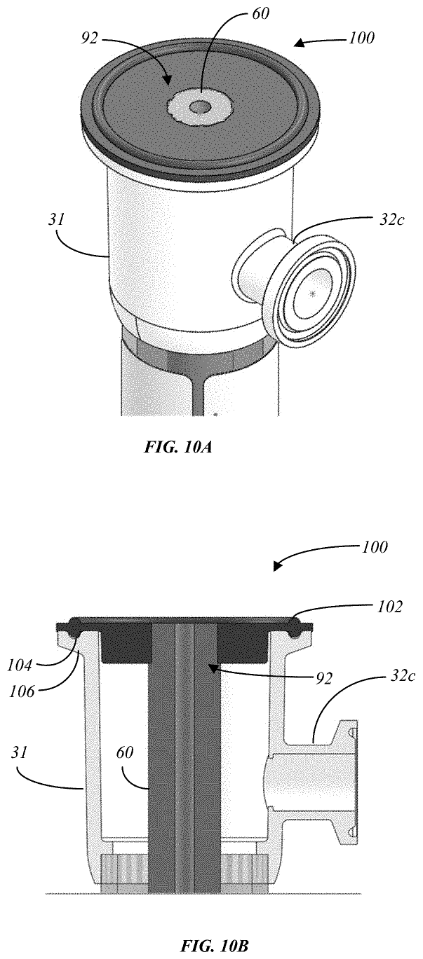

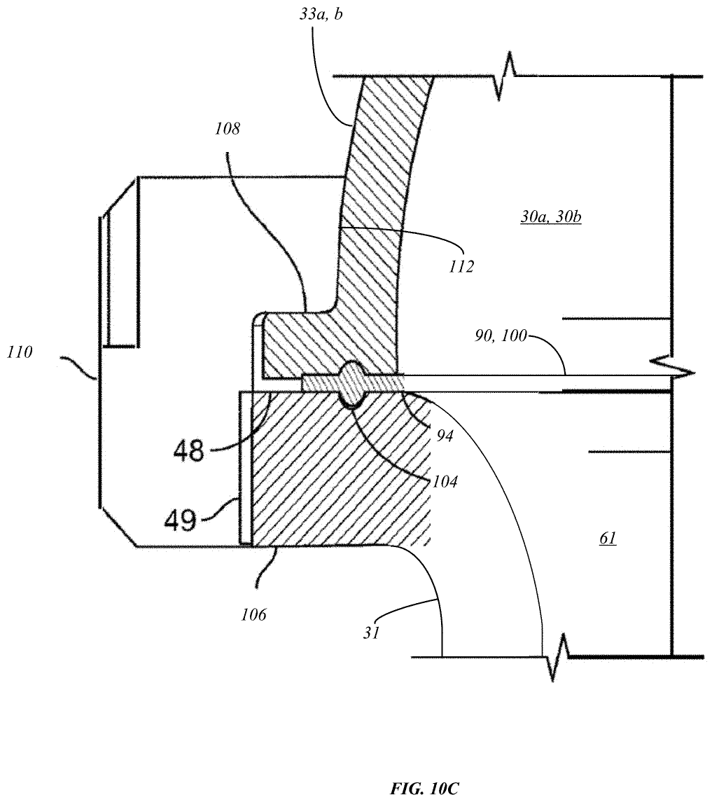

Referring to FIGS. 9A-10C, a removable gasket 90, 100 may encase at least an end of one or more hollow fibers 60, thereby securing the end of the one or more hollow fibers 60 in a position with respect to each other, filter housing 31, or both. For example, gasket 90 comprises a plurality of holes 92 configured to form interference fits with corresponding hollow fibers 60, whereas gasket 100 comprises a single hole 92 configured to fit with a single hollow fiber 60. In various embodiments, one or more holes 92 may comprise a same, a substantially same, or slightly smaller diameter (not shown) than a corresponding hollow fiber to which it is configured to be fit. For example, a hole 92 may comprise a diameter which is 0.1-5.0% smaller than a diameter of a corresponding hollow fiber and be configured to stretch around the diameter when the hollow fiber is extended therethrough. Accordingly, the gasket 90, 100 may be configured to form a fluid-tight fit or otherwise secure fit around one or more hollow fibers 60. Other than the number of hollow fibers accommodated, gaskets 90 and 100 may be understood to comprise one or more similarities to each other as described herein.

Removable gasket 90, 100 may be formed of a single member, for example a molded silicone piece, which may be configured to fit to or couple with a filter housing 31. For example, a peripheral gasket flange 94 may be configured to form an interference fit with filter housing 31 at a corresponding interface such as a ridge 104 of flange 106 of filter housing 31, as illustrated in FIG. 10B. Alternative couplings of gaskets 90, 100 to filter housing 31 are presently contemplated, such as threaded, clipped, luer, glued, welded, clamped, or other fittings (not illustrated for the sake of brevity of the drawings). In some embodiments, as illustrated in FIG. 10C, gasket 90, 100 may be secured to filter housing 31 against a respective end cap 33a, 33b with a clamp ring 110, which in some embodiments may be disposable. For example, end cap 33a, 33b may comprise a peripheral flange 106 to which gasket 90, 100 may be fit.

Flanges 106 and 108 may work together with clamp ring 110 to secure the gasket 90, 100 between the upper and lower surfaces of flanges 106 and 108, respectively. In some embodiments, the flanges 106, 108 may be spaced from each other by a distance somewhat less than the corresponding thickness of gasket flange 94 such that when the two facing peripheral flanges 106, 108 are forced together by clamp ring 110 (e.g., by screwing or otherwise fitting clamp ring 110 about a respectively interfacing outer surface of one or both of flanges 106 and 108), they compress to squeeze gasket flange 94 between the two flanges 106, 108. The compression may create a fluid-tight seal between gasket 90, 100 and filter housing 31 and/or cap 33a, 33b. For example, 10-30% compression or gasket flange 94 may be sufficient to seal both a permeate chamber 61 and respective inlet or outlet chamber 30a, 30b.

In some embodiments, clamp ring 110 may be configured to additionally withstand a high pressure of one or both of permeate chamber 61 and a respective inlet chamber 30a or outlet chamber 30b, for example, by comprising a curved or otherwise non-linear inner wall 112 configured to contact or otherwise accommodate a curved or angled outer surface of filter housing 31 or cap 33a, 33b. Inner wall 112 may thus form a mating surface configured to reinforce a filter housing and/or respective cap 33a, 33b during high pressure conditions. Embodiments are not limited in this context.

It will be recognized that gaskets 90, 100 as described herein may provide improved devices, systems, and/or methods of potting hollow fibers over traditional filtering systems. For example, gasket 90, 100 may double as a sanitary gasket for hollow fiber tangential flow filter unit 30. Removability of gasket 90, 100 may enable quick scaling modifications of filter units. For example, in a down scaling method, gasket 90 and a respective plurality of fitted hollow fibers 60 may be removed from hollow fiber tangential flow filter unit 30 and replaced by an alternative gasket 100 configured to fit with or prefit with fewer hollow fibers 60. Accordingly, a single filter housing may be used for a plurality of operations or methods.

Gasket 90, 100 may be sterilizable and may be either single use or reusable. For example, a single hollow fiber may be replaced from gasket 90, 100, or a plurality of hollow fibers 60 may be replaced from gasket 90, 100, thereby enabling quick and/or easy replacement of hollow fibers 60 of hollow fiber tangential flow filter unit 30. In some embodiments, one or more hollow fibers 60 may be pre-fit and/or permanently coupled to gasket 90, 100. In this example, gasket 90, 100 may be removed from hollow fiber tangential flow filter unit 30 and/or replaced together with fit hollow fiber(s) 60 as a single unit. For example, clamp ring 110 may be removed from about flanges 106, 108, cap 33a, 33b may be removed, and gasket 90, 100 with any coupled hollow fiber(s) 60 may be removed from filter housing 31. In addition to scaling Embodiments are not limited in this context.

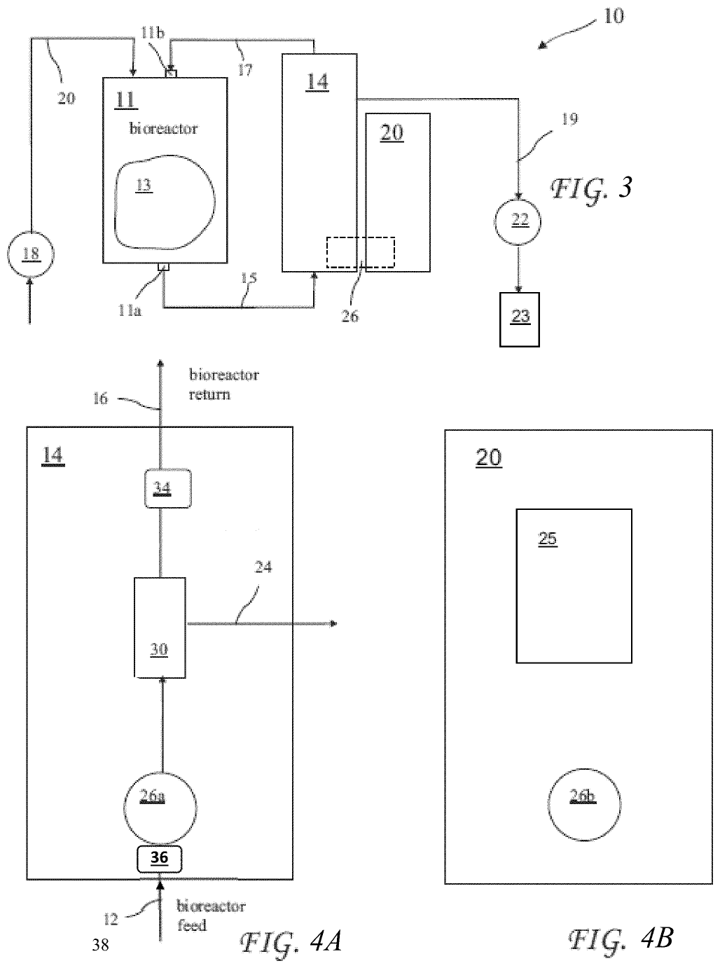

A specific example of a bioreactor system 10 for use in conjunction with the present disclosure will now be described. With reference to FIGS. 3, 4A and 4B, the bioreactor system 10 includes a bioreactor vessel 11 containing bioreactor fluid 13, a tangential flow filtering system 14, and a control system 20. The tangential flow filtering system 14 is connected between a bioreactor outlet 11a and bioreactor inlet l lb to receive bioreactor fluid 12 (also referred to as a bioreactor feed), which contains, for example, cells, cell debris, cell metabolites including waste metabolites, expressed proteins, etc., through bioreactor tubing 15 from the bioreactor 11 and to return a filtered flow 16 (also referred to as a retentate flow or bioreactor return) through return tubing 17 to the bioreactor 11. The bioreactor system 10 cycles bioreactor fluid through the tangential flow filtering system 14 which removes various materials (e.g., cell debris, soluble and insoluble cell metabolites and other products produced by cells including expressed proteins, viruses, virus like particles (VLPs), exosomes, lipids, DNA, or other small particles) from the bioreactor fluid and returns cells to allow the reaction in the bioreactor vessel 11 to continue. Removing waste metabolites allows the continued proliferation of cells within the bioreactor, thereby allowing the cells to continue to express recombinant proteins, antibodies or other biological materials that are of interest.

The bioreactor tubing 15 may be connected, for example, to the lowest point or dip tube of the bioreactor 11 and the return tubing 17 may be connected to the bioreactor 11, for example, in the upper portion of the bioreactor volume and submerged in the bioreactor fluid 13.

The bioreactor system 10 includes an assembly comprising a hollow fiber tangential flow filter unit 30 (described in more detail above), a pump 26, and associated fittings and connections. Any suitable pump may be used in conjunction with the present disclosure including, for example, peristaltic pumps, positive displacement pumps, and pumps with levitating rotors inside the pumpheads, among others. As a specific example, the pump 26 may include a low shear, gamma-radiation stable, disposable, levitating pumphead 26a, for example, a model number PURALEV® 200SU low shear re-circulation pump manufactured by Levitronix, Waltham, Mass., USA. The PURALEV® 200SU includes a magnetically levitated rotor inside a disposable pumphead, and stator windings in the pump body, allowing simple removal and replacement of the pumphead 26a.

The flow of bioreactor fluid 12 passes from the bioreactor vessel 11 to the tangential flow filtering system 14 and the return flow of the bioreactor fluid 16 passes from the tangential flow filtering system 14 back to the bioreactor vessel 11. A permeate flow 24 (e.g., containing soluble and insoluble cell metabolites and other products produced by cells including expressed proteins, viruses, virus like particles (VLPs), exosomes, lipids, DNA, or other small particles, or containing cells or particles having a diameter below a threshold value, or both) is stripped from the flow of bioreactor material 12 by the tangential flow filtering system 14 and carried away from the tangential flow filtering system 14 by tubing 19. The permeate flow 24 is drawn from the hollow fiber tangential flow system 14 by a permeate pump 22 into a storage container 23.

In the embodiment shown, the tangential flow filtering system 14 (see FIG. 4A) includes a disposable pumphead 26a, which simplifies initial set up and maintenance. The pumphead 26a circulates the bioreactor fluid 12 through the hollow fiber tangential flow filter unit 30 and back to the bioreactor vessel 11. A non-invasive transmembrane pressure control valve 34 may be provided in line with the flow 16 from the hollow fiber tangential flow filter unit 30 to the bioreactor vessel 11, to control the pressure within the hollow fiber tangential flow filter unit 30. For example, the valve 34 may be a non-invasive valve, which resides outside tubing carrying the return flow 16 that squeezes the tubing to restrict and control the flow, allowing the valve to regulate the applied pressure on the membrane. Alternatively, or in addition, a flow controller 36 may be provided at the pumphead 26a inlet in order to provide pulsed flow to the hollow fiber tangential flow filter unit 30, as described in more detail below. The permeate flow 24 may be continually removed from the bioreactor fluid 13 which flows through the hollow fiber tangential flow filter unit 30. The pumphead 26a and the permeate pump 22 are controlled by the control system 20 to maintain the desired flow characteristics through the hollow fiber tangential flow filter unit 30.

The pumphead 26a and hollow fiber tangential flow filter unit 30 in the tangential flow filtering system 14 may be connected by flexible tubing allowing easy changing of the elements. Such tubing allows aseptic replacement of the hollow fiber tangential flow filter unit 30 in the event that the hollow fiber tangential flow filter unit 30 becomes plugged with material and therefore provides easy exchange to a new hollow fiber assembly.

The tangential flow filtering system 14 may be sterilized, for example, using gamma irradiation, ebeam irradiation, or ETO gas treatment.

Referring again to FIG. 1, during operation, two permeate fluid outlet ports 32b and 32c may be employed to remove permeate flow 24 in some embodiments. In other embodiments, only a single permeate fluid outlet port may be employed. For example, permeate flow 24 may be collected from the upper permeate port 32c only (e.g., by closing permeate port 32b) or may be collected from the lower permeate port 32b only (e.g., by draining the permeate flow 24 from the lower permeate port 32b while the permeate port 32c closed or kept open). In certain beneficial embodiments, the permeate flow 24 may be drained from the lower permeate port 32b to reduce or eliminate Sterling flow, which is a phenomenon where an upstream (lower) end of the of the hollow fibers 60 (the high-pressure end) generates permeate that back-flushes the downstream (upper) end of the hollow fibers 60 (the low-pressure end). Draining the permeate flow 24 from the lower permeate port 32b leaves air in contact with the upper end of the of the hollow fibers 60 minimizing or eliminating Sterling flow.

In certain embodiments, the bioreactor fluid 12 may be introduced into the hollow fiber tangential flow filter unit 30 at a constant flow rate.

In certain embodiments, the bioreactor fluid may be introduced into the hollow fiber tangential flow filter unit 30 in a pulsatile fashion (i.e., under pulsed flow conditions), which has been shown to increase permeate rate and volumetric throughput capacity. As used herein “pulsed flow” is a flow regime in which the flow rate of a fluid being pumped (e.g., fluid entering the hollow fiber tangential flow filter unit) is periodically pulsed (i.e., the flow has periodic peaks and troughs). In some embodiments, the flow rate may be pulsed at a frequency ranging from 1 cycle per minute or less to 2000 cycles per minute or more (e.g., ranging from 1 to 2 to 5 to 10 to 20 to 50 to 100 to 200 to 500 to 1000 to 2000 cycles per minute) (i.e., ranging between any two of the preceding values). In some embodiments, the flow rate associated with the troughs is less than 90% of the flow rate associated with the peaks, less than 75% of the flow rate associated with the peaks, less than 50% of the flow rate associated with the peaks, less than 25% of the flow rate associated with the peaks, less than 10% of the flow rate associated with the peaks, less than 5% of the flow rate associated with the peaks, or even less than less than 1% of the flow rate associated with the peaks, including zero flow and periods of backflow between the pulses. In various embodiments, the flow rate may be pulsed at a frequency at least 2000, 2300, 2500, 3000, 3500 or 4000 times greater than the quotient of the kinematic viscosity over the TDF inner diameter, as described above.

Pulsed flow may be generated by any suitable method. In some embodiments, pulsed flow may be generated using a pump such as a peristaltic pump that inherently produces pulsed flow. For example, tests have been run by applicant which show that switching from a pump with a magnetically levitated rotor like that described above under constant slow conditions to a peristaltic pump (which provides a pulse rate of about 200 cycles per minute) increases the amount of time that a tangential flow depth filter can be operated before fouling (and thus increases the quantity of permeate that can be collected).

In some embodiments, pulsed flow may be generated using pumps that otherwise provide a constant or essentially constant output (e.g., a positive displacement pump, centrifugal pumps including magnetically levitating pump, etc.) by employing a suitable flow controller to control the flow rate. Examples of such flow controllers include those having electrically controlled actuators (e.g. a servo valve or solenoid valve), pneumatically controlled actuators or hydraulically controlled actuators to periodically restrict fluid entering or exiting the pump. For example, in certain embodiments, a flow controller 36 may be placed upstream (e.g., at the inlet) or downstream (e.g., at the outlet) of a pump 26 like that described hereinabove (e.g., upstream of pumphead 26a in FIG. 4A) and controlled by a controller 20 to provide pulsatile flow having the desired flow characteristics.

In other embodiments, pulsed flow may be adjusted throughout a filtering cycle, for example, based on a viscosity measurement of bioreactor fluid 13. For example, controller 36 may adjust a pulsed flow based on a measurement by a viscosity sensor 38. Controller 36 may be manually adjustable based on the measurement by viscosity sensor 38, or controller 36 may be communicatively coupled to viscosity sensor 36 so as to automatically adjust the pulsed flow based on the measurement thereby, for example, during a dynamic flow of bioreactor feed 12.

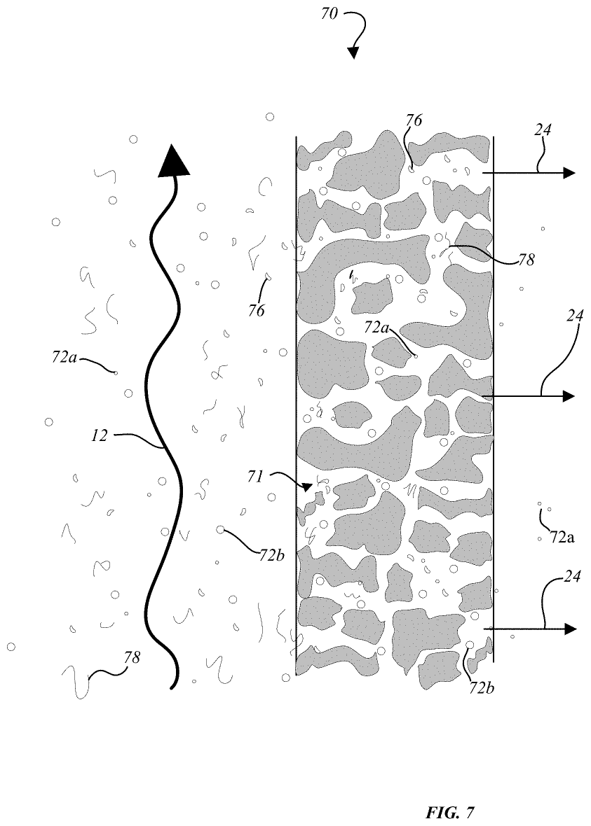

For example, as illustrated in FIG. 7, bioreactor feed 12 may be under turbulent or non-laminar flow, which may prevent culture medium contents such as lysed cells 76 and/or extracellular material 78, such as DNA, from fouling wall 70. Lysed cells 76 may comprise mammalian cells, for example, HEK293 or CHO cells, or other cell type, such as bacterial cells, any or each of which may be appropriate in harvesting applications of plasmid DNA, intracellular proteins, or the like. In some embodiments, the thick wall 70 may capture and/or accommodate some of lysed cells 76 and/or extracellular material 78, and in some embodiments may pass through the same into permeate 24 (lysed cells 76 and extracellular material 78 not shown in bioreactor fluid 12), thereby decreasing a turbidity of bioreactor fluid 12. As described with respect to FIGS. 4A-4C above, a controller 36 may then adjust a flow rate of bioreactor fluid 12 based on the change in viscosity thereof.

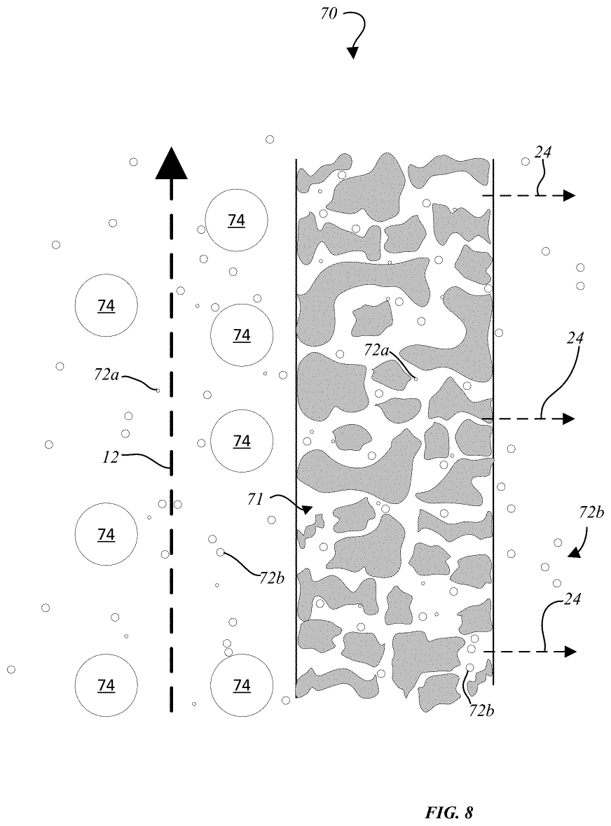

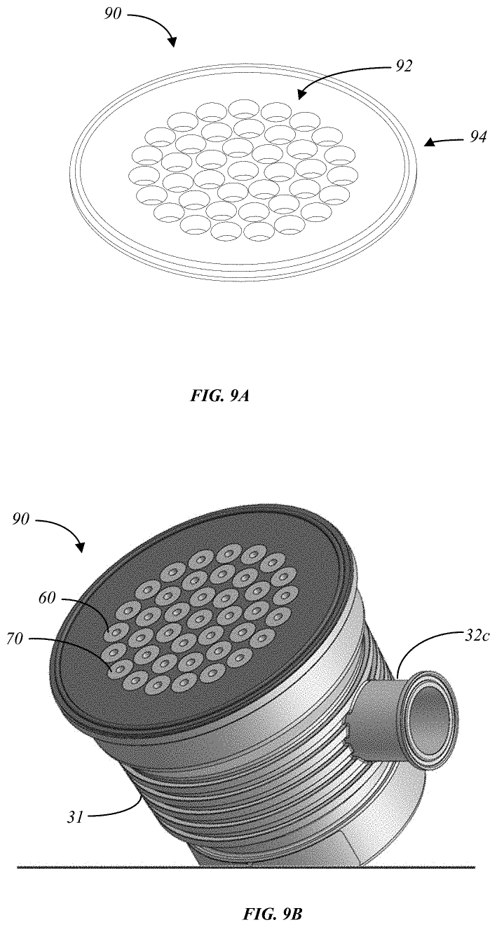

In another example, as illustrated in FIG. 8, bioreactor feed 12 may be under laminar or non-laminar flow (not shown). Large pores of wall 70 may allow intermediate-sized particles 72b, which may be, for example, cells ranging in diameter from 5 microns to 150 microns, to pass therethrough. Meanwhile, wall 70 may prevent large particles 74 such as microcarriers or cell clumps from passing therethrough. In some examples, filament(s) used to form wall 70 may comprise a charge and/or affinity ligand which may trap one or more small particles 72a within wall 70, allowing a selective permeate comprising only intermediate-sized particles. It will be recognized by one of skill in the art that such a configuration may be useful for selectively harvesting cells, for example, in a sorting process. For example, individual cells, such as Chinese Hamster Ovary (CHO) cells, may be shed from cell clumps in bioreactor fluid 12 as they differentiate, and they may then be passed through wall 70. In some embodiments, selective removal of differentiating cells via TDF system may decrease a turbidity and/or biological load of bioreactor fluid 12 so as to promote increased or maintained growth rates within bioreactor fluid 12. Embodiments are not limited in this context.

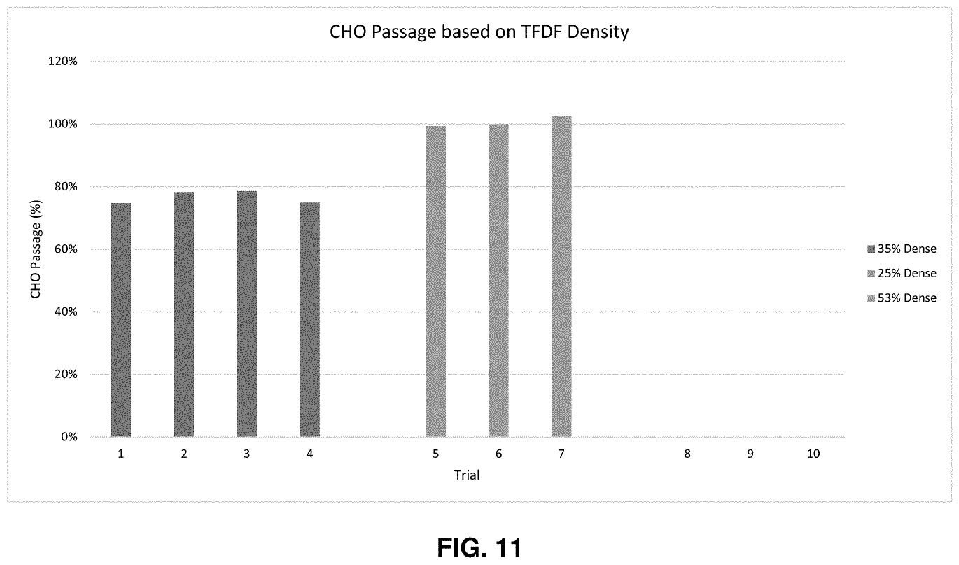

In an embodiment illustrated by FIG. 11, CHO cells at ˜60E6 VCD were passed through a TDF system with TFDF filters of varying density. The results shown in the bar graph of FIG. 11 indicate that the 35% dense filter (Trials 1-4) passes about 80% of the cells into the permeate, the 25% dense filter (Trials 5-7) passes 100% of the cells and the 53% dense filter effectively passes no cells (Trials 8-10). All three TFDF filter retain. Additionally, the all three filters retained 150 μm polystyrene latex beads. Since CHO cells are approximately 15 μm, the results show that the 25-35% density filters would be sufficient to pass individual mammalian cells while retaining larger particles such as microcarriers or cell clumps.

It will be understood that various examples discussed above are non-limiting, and that any combination of concepts discussed herein is within the scope of the present disclosure.

1. A hollow fiber tangential flow filter unit for bioprocessing applications comprising:
(a) a housing having an interior,
(b) a fluid inlet,
(c) a retentate fluid outlet,
(d) a permeate fluid outlet, and
(e) at least one hollow fiber comprising a porous wall, said fiber being formed from a plurality of extruded polymer filaments, wherein the at least one hollow fiber has an interior surface and an exterior surface, the interior surface forming an interior lumen having a width ranging from about 0.75 mm to about 30.0 mm and extending through the at least one hollow fiber, and the at least one hollow fiber having a mean pore size ranging from about 5 microns to about 150 microns,
wherein the at least one hollow fiber is positioned in the housing interior, wherein the fluid inlet and the retentate fluid outlet are in fluid communication with the interior lumen of the at least one hollow fiber and wherein the permeate fluid outlet is in fluid communication with the housing interior and the exterior surface of the porous wall.

2. The hollow fiber tangential flow filter unit of claim 1, wherein the extruded polymer filaments are mono-component filaments or bi-component filaments.

3. The hollow fiber tangential flow filter unit of claim 1, wherein the at least one hollow fiber comprises two or more layers of varying density.

4. The hollow fiber tangential flow filter unit of claim 1, wherein the mean pore size ranges from about 15 microns to about 80 microns, from about 20 microns to about 60 microns, or from about 30 microns to about 50 microns.

5. The hollow fiber tangential flow filter unit of claim 1, wherein the density of the hollow fiber is about 10% to about 40%, or is about 15%, about 20%, about 25%, about 30%, or about 35% of the density of an equivalent solid volume fiber.

6. The hollow fiber tangential flow filter unit of claim 1, wherein the fiber has a wall thickness ranging from about 1 mm to about 10 mm, from about 2 mm to about 3 mm, from about 2 mm to about 10 mm, from about 5 to about 10 mm, or is about 5 mm.

7. The hollow fiber tangential flow filter unit of claim 1, wherein the interior lumen has a width ranging from about 1 mm to about 2 mm, from about 2 mm to about 10 mm, from about 5 mm to about 20 mm, or is about 5 mm.

8. The hollow fiber tangential flow filter unit of claim 1, comprising a plurality of hollow fibers.

9. The hollow fiber tangential flow filter unit of claim 8, wherein the hollow fiber tangential flow filter unit is configured to couple with a silicone potting gasket.

10. The hollow fiber tangential flow filter unit of claim 1, wherein said filter is a hollow fiber tangential flow depth filter.

11. A hollow fiber tangential flow filtering system comprising:
(a) a hollow fiber tangential flow filter unit of claim 1; and
(b) a pumping system configured to provide pulsed flow to the fluid inlet.

12. The hollow fiber tangential flow filtering system of claim 11, wherein the at least one hollow fiber comprises extruded mono-component filaments or extruded bi-component filaments.

13. The hollow fiber tangential flow filtering system of claim 11, wherein the at least one hollow fiber comprises two or more layers of varying density.

14. The hollow fiber tangential flow filtering system of claim 11, wherein the mean pore size ranges from about 15 microns to about 80 microns, from about 20 microns to about 60 microns, or from about 30 microns to about 50 microns.

15. The hollow fiber tangential flow filter system of claim 11, wherein the density of the hollow fiber is about 10% to about 40%, or is about 15%, about 20%, about 25%, about 30%, or about 35% of the density of an equivalent solid volume fiber.

16. The hollow fiber tangential flow filter system of claim 11, wherein the fiber has a wall thickness ranging from about 1 mm to about 10 mm, or from about 2 mm to about 3 mm, or from about 2 mm to about 8 mm, or is about 5 mm.

17. The hollow fiber tangential flow filter system of claim 11, wherein the interior lumen has a width ranging from about 1 mm to about 2 mm, or from about 2 mm to 10 mm, or from about 5 mm to about 20 mm, or is about 5 mm.

18. The hollow fiber tangential flow filter system of claim 11, comprising a plurality of said hollow fibers.

19. The hollow fiber tangential flow filter system of claim 18, wherein the hollow fiber tangential flow filter unit is configured to couple with a silicone potting gasket.

20. The hollow fiber tangential flow filtering system o of claim 11, wherein said filter is a hollow fiber tangential flow depth filter.

21. The hollow fiber tangential flow filtering system of claim 11, wherein the pumping system comprises a pulsatile pump.

22. The hollow fiber tangential flow filtering system of claim 11, wherein the pumping system comprises a flow controller capable of providing and regulating pulsed flow.

23. The hollow fiber tangential flow filtering system of claim 22, wherein the flow controller comprises an actuator selected from the group consisting of an electrically-controlled actuator, a pneumatically-controlled actuator or a hydraulically-controlled actuator.

24. The hollow fiber tangential flow filtering system of claim 22, wherein said flow controller is configured to provide a pulsed flow at a rate determined by a change in viscosity when fluid is present in said system.

25. The hollow fiber tangential flow depth filtering system of claim 24, further comprising a viscosity sensor.

26. The hollow fiber tangential flow filtering system of claim 25 further comprising a viscosity sensor communicatively coupled to a second controller, wherein said second controller is configured to adjust the rate of the pulsed flow based on a measurement of the viscosity sensor.

27. The hollow fiber tangential flow filtering system of claim 11, wherein said system is a hollow fiber tangential flow depth filter system.

28. A method of filtration which comprises
(a) introducing a fluid comprising large size particles and small size particles into the fluid inlet of a hollow fiber tangential flow filter unit of claim 1 under tangential flow conditions suitable to separate said fluid into a permeate comprising the small particles and a retentate comprising the large particles; and
(b) collecting said permeate through the permeate fluid outlet and said retentate through the retentate fluid outlet.

29. The method of claim 28, wherein the fluid further comprises intermediate-sized particles and wherein at least a portion of the intermediate-sized particles are trapped in the wall of the at least one hollow fiber.

30. The method of claim 28, wherein the large particles comprise microcarriers or cell clumps.

31. The method of claim of claim 28, wherein the small particles comprise one or more of proteins, cells, viruses, virus-like particles (VLPs), exosomes, lipids, DNA, and cell metabolites.

32. The method of claim 28, wherein the large particles, the small particles and the intermediate-sized particles are selected from ceramic particles, metal particles, liposomal structures for drug delivery, biodegradable polymeric particles, and microcapsules.

33. The method of claim 28, wherein said fluid is pumped at a pulsed flow rate based on viscosity of the fluid.

34. A method of harvesting biological matter from a culture which comprises
(a) introducing a continuous culture feed into the fluid inlet of a hollow fiber tangential flow filter unit of claim 1 at a flow rate based on the viscosity of said culture feed before entry thereof into said fluid inlet and the diameter of the interior lumen of the hollow fiber;
(b) flowing said culture feed through said filter to obtain a permeate comprising the biological matter for harvest and a retentate;
(c) collecting said permeate through the permeate fluid outlet and said retentate through the retentate fluid outlet; and
(d) harvesting said biological matter from the permeate.

35. The method of claim 34, wherein the culture feed is a bioreactor feed.

36. The method of claim 34 which comprises measuring viscosity of the retentate and adjusting the flow rate of said culture feed based on the retentate viscosity
